(19) United States
(12) Patent Application Publication
(10) Pub. No.: US 2026/0274990 A1
(43) Pub. Date: Sep. 17, 2026

(54) TROGOCYTOSIS IN CANCER CELLS AND METHODS FOR TREATING CANCER RELATED THERETO

(71) Applicant: ARIZONA BOARD OF REGENTS ON BEHALF OF THE UNIVERSITY OF ARIZONA, Tucson, AZ (US)

(72) Inventors: Alfred BOTHWELL, Tucson, AZ (US); Haley MARCARIAN, Tucson, AZ (US); Anutr SIVAKOSES, Tucson, AZ (US)

(21) Appl. No.: 19/472,708

(22) PCT Filed: Apr. 3, 2024
(86) PCT No.: PCT/US2024/022840

§ 371 Date: Oct. 6, 2025

Related U.S. Application Data
(60) Provisional application No. 63/494,365, filed on Apr. 5, 2023.

(51) Int. Cl. C07K 16/40; A61K 47/68; A61P 35/00; C07K 16/28; G01N 33/50

(52) U.S. Cl. CPC C07K 16/40; A61K 47/68031; A61K 47/68033; A61K 47/6809; A61K 47/6849; A61K 47/6871; A61P 35/00; C07K 16/2803; C07K 16/283; C07K 16/289; C07K 16/2896; C12Y 402/01001; G01N 33/5011; C07K 2317/31

(57) ABSTRACT

It has been discovered that cancer cells obtain immune cell markers by trogocytosis. Thus, methods of identifying and characterizing non-immune cells by the presence of immune cell markers thereon are provided. The method can indicate that the non-immune cells (e.g., cancer cells) have undergone trogocytosis. Such a determination can be used to design and guide cancer treatment. Thus, compositions and methods for selectively targeting and/or killing cancer cells in a subject have also been developed. In some embodiments, the compositions include an antibody, preferably a bispecific antibody, that selectively targets tumor cells with an antigen binding domain that targets an immune cell marker present on cancer cells, preferably, one that is deposited from immune cells onto tumor cell surface. In additional/alternative embodiments, the compositions and methods include administering a subject a composition for inhibiting trogocytosis. In some forms, inhibition of trogocytosis enhances the therapeutic efficacy of existing cancer therapies.

### CROSS-REFERENCE TO RELATED APPLICATIONS

This application claims the benefit of and priority to U.S. Application No. 63/494,365 filed Apr. 5, 2023, the contents of which are incorporated by reference herein in their entirety.

### FIELD OF THE INVENTION

The invention is generally in the field of biologics for treating cancer and more particularly to agents that selectively target cancer cells having undergone trogocytosis.

### BACKGROUND OF THE INVENTION

Trogocytosis is the transfer of membrane fragments from a donor cell to an acceptor cell (Joly, E., Nature Immunology, 2003. 4(9): p. 815). In mammalian cells, this process was first described on CD8+ T cells, in which the CD8+ T cells were shown to take fragments of the plasma membrane from certain antigen presenting cells (APCs) during the formation of the immunological synapse (Arnold, P. Y., D. K. Davidian, and M. D. Mannie, European Journal of Immunology, 1997. 27(12): p. 3198-3205). Since then, trogocytosis has been observed in many different types of immune cells, including CD4+ T cells (Osborne, D. G. and S. A. Wetzel, J Immunol, 2012. 189(10): p. 4728-39), Natural Killer (NK) cells (Nakayama, M., et al., Proc Natl Acad Sci USA, 2011. 108(45): p. 18360-5), and macrophages (Sarvari, A. K., et al., Cell Death Dis, 2015. 6(1): p. e1613). Previously, it was shown that trogocytosis plays an important role in the acquisition of immune regulatory molecules by colon cancer cells from infiltrating lymphocytes (Shin, J. H., et al., Proc Natl Acad Sci USA, 2021. 118(48)). This resulted in increased immune regulator molecules such as CTLA4, PDL1, Tim3, VISTA, LAG3, CD38, CD80, CD86, MHC Class II, and PD-L1 being presented on the surface of trogocytic tumor cells compared to non-trogocytic tumor cells. Trogocytosis was also observed with numerous lymphocyte populations including macrophages, dendritic cells, NK cells, and monocytes. Along with the transfer of surface proteins, genomic DNA PCR analysis also showed a transfer of DNA from donor immune cells to mouse CRC cells, implying that tumor cells can display an altered transcriptome because of trogocytosis. Trogocytosis has also been implicated in other types of disease such as Hodgkin lymphoma and renal cell carcinoma (Zeng, Q., and H. Schwarz, Oncoimmunology, 2020. 9(1): p. 1781334; Park, H. R., et al., Sci Rep, 2022. 12(1): p. 12546). In mouse models of leukemia, trogocytosis has been shown to promote the transfer of target antigens from cancer cells to T cells, which can induce T cell exhaustion and fratricide (Hamieh, M., et al., Nature, 2019. 568(7750): p. 112-116).

Trogocytosis is involved in attenuating therapeutic efficacy in tumor therapies. Some studies have revealed that FcγR-mediated trogocytosis by immune cells reduces the effects of a variety of monoclonal antibody (mAb)-based therapies (Levchenko A, et al., Proc Natl Acad Sci USA (2005) 102(6):1933-8; Beum P V, et al., J Immunol (2006) 176(4):2600-9; Williams M E, et al. J Immunol (2006) 177(10):7435-43). This is a particular concern when treating patients with CAR T cell therapies and is a common cause of relapses in individuals undergoing this type of treatment.

Therefore, there is a need for additional cancer therapeutics with enhanced anti-cancer therapeutic efficacy.

Therefore, it is an object of the invention to provide alternative cancer therapeutics optionally having improved anti-tumor efficacy and methods thereof for treating proliferative diseases.

It is another object of the invention to provide compositions and methods for reducing or inhibiting trogocytosis.

It is another object of the invention to detect cancer cells that have undergone trogocytosis, optionally in combination with using this information to characterize the cells and select or monitor therapies directed thereto.

### SUMMARY OF THE INVENTION

Methods of characterization of non-immune cells (e.g., cancer cells) including detecting one or more immune cell markers on non-immune cells of a sample from a subject (e.g., a biopsy) are provided. In some embodiments, the non-immune cells are suspected or known to be cancer cells. Thus, methods of diagnosing and prognosing cancer are also provided. The methods can further include detecting one or more cancer antigens that are not immune cell marker(s). Detection can include, for example, an immunoassay utilizing an antibody or antibodies specific to the immune cell marker and/or cancer antigen. In some embodiments, detection of the immune cell marker(s) indicates the cancer cells were subject to trogocytosis. In embodiments, the immune cell marker(s) is CD45 optionally CD45RA and/or CD45RO, CD14, CD16, CD56, CD68, CD4, CD53, PTPRC, FCGR2A, FCGR3A, and/or CTSS.

Compositions and methods of treating trogocytosis and cancer are also provided and can be used alone or in combination with the methods of detection, characterization, and diagnosis. In some embodiments, the compositions include an antibody or antigen binding fragment that specially binds to an immune cell antigen present on a cancer cell, optionally wherein presence or expression of the immune cell antigen on the cancer cell is due trogocytosis. Thus provided are compositions and methods of use thereof that target immune cell markers. For example, provided are antibodies and antigen binding fragments including i) a first antigen-binding site, and ii) a second antigen-binding site, wherein the first antigen-binding site specifically binds to an immune cell antigen present on a cancer cell, and the second antigen-binding site specifically binds to a non-immune cell cancer antigen, optionally wherein presence or expression of the immune cell antigen on the cancer cell is due to trogocytosis.

Also provided are antibodies and antigen binding fragments including i) a first antigen-binding site, and ii) a second antigen-binding site, wherein the first antigen-binding site specifically binds to an immune cell antigen present on a cancer cell, and the second antigen-binding site specifically binds to an antigen on an immune cell, optionally wherein presence or expression of the immune cell antigen on the cancer cell is due to trogocytosis, optionally wherein the cancer cell does not have the antigen on the immune cell.

Any of the antibodies and fragments can be bispecific, trispecific, or multispecific. In particular embodiments, the antibody or fragment is a Bispecific T cell engager (BiTE). In some embodiments, the immune cell antigen present on the cancer cell is a cell surface molecule selected from the group consisting of CD45, CD56, CD16, CD14, CD68, CD4, CD53, PTPRC, FCGR2A, FCGR3A, and CTSS. Examples of other cancer antigens and immune cells markers that can be targeted by the antibodies and antigen binding fragments are also provided. Any of the antibodies or antigen binding fragments can be conjugated to one of more of cytotoxic agents, drugs, receptors, enzymes, receptor ligands. Cytotoxic agents can be, for example, anti-microtubules agents, DNA-damaging agents, maytansinoids, auristatins, or calicheamicins. In some embodiments, the antibody or antigen binding fragment includes an Fc fragment that facilitates binding to a Fc receptor (e.g., FcγR) on an immune effector cell such as NK cells, monocytes, macrophages, neutrophils, eosinophils, or dendritic cells. In some embodiments, binding to Fc receptor facilitates cytotoxic granule release, Fas signaling, elaboration of reactive oxygen species, or combinations thereof. Thus, the antibodies and antigen binding fragments can be antibody-drug conjugates and/or have antibody-dependent cell-mediated cytotoxicity (ADCC) activity, an altered C1q binding activity, an altered complement-dependent cytotoxicity activity (CDC), a phagocytic activity, or any combination thereof.

In additional and alternative embodiments, the composition can be or include one that inhibits trogocytosis. Such composition can be or include one or more Gal/GalNAc (D-galactose/N-acetyl-D-galactosamine) lectin inhibitors, PI3K/AKT/mTOR pathway inhibitors, amoebic AGC kinase 1 (EhAGCK1) inhibitors, tyrosine-protein kinase Syk inhibitors, ATPase inhibitors, and/or combinations thereof.

Pharmaceutical composition including a therapeutically effective amount of the disclosed compositions are also provided, and can be formulated for, e.g., intravenous, intrathecal, intratumoral, or oral administration. In particular embodiments the pharmaceutical compositions are formulated for local or topical administration.

The disclosed methods of treatment typically include administering to a subject in need thereof an effective amount of a composition, e.g., a pharmaceutical composition. Any of the compositions and methods can further include treating the subject with surgery, a radiation therapy, chemotherapy, and/or immunotherapy optionally inhibition of one or more checkpoint proteins and/or adoptive cell therapy. The cancer can be, for example, breast cancer, head and neck squamous cell carcinoma, glioblastoma, pancreatic cancer, lung cancer, melanoma, ovarian cancer, or sarcoma. In a particular embodiment, the cancer is a kidney cancer such as renal cell carcinoma. In some embodiments, the cancer was resistant or insensitive to one or more conventional chemotherapeutic agents and/or immunotherapy, prior to the administration of the pharmaceutical composition.

Any of the methods can further include detecting the immune cell marker(s) in one or more subsequent samples taken after a treatment. In such embodiments, a reduction of the immune cell marker(s) over time is an indication that the treatment is effective.

Methods of identifying a compound that reduces trogocytosis and/or treats cancer including detecting one or more immune cell markers on cancer cells prior and after treatment with a test compound are also provided. The compound can be selected as effective for reducing trogocytosis and/or treating cancer if the immune cell markers is reduced after treatment relative to before treatment. The treatment can be in vivo, in vitro, or ex vivo.

Methods of characterization of non-immune cells (e.g., cancer cells) including detecting one or more immune cell markers on non-immune cells of a sample from a subject (e.g., a biopsy) are provided. In some embodiments, the non-immune cells are suspected or known to be cancer cells. Thus, method of diagnosing and prognosing cancer are also provided. The methods can further include detecting one or more cancer antigens that are not immune cell marker(s). Detection can include, for example, an immunoassay utilizing an antibody or antibodies specific to the immune cell marker and/or cancer antigen. In some embodiments, detection of the immune cell marker(s) indicates the cancer cells were subject to trogocytosis. In embodiments, the immune cell marker(s) is CD45 optionally CD45RA and/or CD45RO, CD14, CD16, CD56, CD68, CD4, CD53, PTPRC, FCGR2A, FCGR3A, and/or CTSS.

Compositions and methods of treating trogocytosis and cancer are also provided and can be used alone or in combination with the methods of detection, characterization, and diagnosis. In some embodiments, the compositions include an antibody or antigen binding fragment that specially binds to an immune cell antigen present on a cancer cell, optionally wherein presence or expression of the immune cell antigen on the cancer cell is due trogocytosis. Thus provided are compositions and methods of use thereof that target immune cell markers. For example, provided are antibodies and antigen binding fragments including i) a first antigen-binding site, and ii) a second antigen-binding site, wherein the first antigen-binding site specifically binds to an immune cell antigen present on a cancer cell, and the second antigen-binding site specifically binds to a non-immune cell cancer antigen, optionally wherein presence or expression of the immune cell antigen on the cancer cell is due to trogocytosis.

Also provided are antibodies and antigen binding fragments including i) a first antigen-binding site, and ii) a second antigen-binding site, wherein the first antigen-binding site specifically binds to an immune cell antigen present on a cancer cell, and the second antigen-binding site specifically binds to an antigen on an immune cell, optionally wherein presence or expression of the immune cell antigen on the cancer cell is due to trogocytosis, optionally wherein the cancer cell does not have the antigen on the immune cell.

Any of the antibodies and fragments can be bispecific, trispecific, or multispecific. In particular embodiments, the antibody or fragment is a Bispecific T cell engager (BiTE). In some embodiments, the immune cell antigen present on the cancer cell is a cell surface molecule selected from the group consisting of CD45, CD56, CD16, CD14, CD68, CD4, CD53, PTPRC, FCGR2A, FCGR3A, and CTSS. Examples of other cancer antigens and immune cells markers that can be targeted by the antibodies and antigen binding fragments are also provided. Any of the antibodies or antigen binding fragments can be conjugated to one of more of cytotoxic agents, drugs, receptors, enzymes, receptor ligands. Cytotoxic agents can be, for example, anti-microtubules agents, DNA-damaging agents, maytansinoids, auristatins, or calicheamicins. In some embodiments, the antibody or antigen binding fragment includes an Fc fragment that facilitates binding to a Fc receptor (e.g., FcγR) on an immune effector cell such as NK cells, monocytes, macrophages, neutrophils, eosinophils, or dendritic cells. In some embodiments, binding to Fc receptor facilitates cytotoxic granule release, Fas signaling, elaboration of reactive oxygen species, or combinations thereof. Thus, the antibodies and antigen binding fragments can be antibody-drug conjugates and/or have antibody-dependent cell-mediated cytotoxicity (ADCC) activity, an altered C1q binding activity, an altered complement-dependent cytotoxicity activity (CDC), a phagocytic activity, or any combination thereof.

In additional and alternative embodiments, the composition can be or include one that inhibits trogocytosis. Such composition can be or include one or more Gal/GalNAc (D-galactose/N-acetyl-D-galactosamine) lectin inhibitors, PI3K/AKT/mTOR pathway inhibitors, amoebic AGC kinase 1 (EhAGCK1) inhibitors, tyrosine-protein kinase Syk inhibitors, ATPase inhibitors, and/or combinations thereof.

Pharmaceutical composition including a therapeutically effective amount of the disclosed compositions are also provided, and can be formulated for, e.g., intravenous, intrathecal, intratumoral, or oral administration. In particular embodiments the pharmaceutical compositions are formulated for local or topical administration.

The disclosed methods of treatment typically include administering to a subject in need thereof an effective amount of a composition, e.g., a pharmaceutical composition. Any of the compositions and methods can further include treating the subject with surgery, a radiation therapy, chemotherapy, and/or immunotherapy optionally inhibition of one or more checkpoint proteins and/or adoptive cell therapy. The cancer can be, for example, breast cancer, head and neck squamous cell carcinoma, glioblastoma, pancreatic cancer, lung cancer, melanoma, ovarian cancer, or sarcoma. In a particular embodiment, the cancer is a kidney cancer such as renal cell carcinoma. In some embodiments, the cancer was resistant or insensitive to one or more conventional chemotherapeutic agents and/or immunotherapy, prior to the administration of the pharmaceutical composition.

Any of the methods can further include detecting the immune cell marker(s) in one or more subsequent samples taken after treatment. In such embodiments, a reduction of the immune cell marker(s) over time is an indication that the treatment is effective.

Methods of identifying a compound that reduces trogocytosis and/or treats cancer including detecting one or more immune cell markers on cancer cells prior and after treatment with a test compound are also provided. The compound can be selected as effective for reducing trogocytosis and/or treating cancer if the immune cell markers is reduced after treatment relative to before treatment. The treatment can be in vivo, in vitro, or ex vivo.

### BRIEF DESCRIPTION OF THE DRAWINGS

FIG. 1A is a graph showing average percentage of trogocytic tumor cells for 21 human RCC tumors expressing CD14, CD16, CD56, or CD45RA. FIGS. 1B-1E are graphs showing average percentage of trogocytic tumor cells in 21 human RCC tumors organized by tumor stage expressing CD56 (FIG. 1), CD16 (FIG. 1C), CD45RA (FIG. 1D), and CD14 (FIG. 1E).

FIG. 2A is flow cytometry analysis of trogocytosis in fresh RCC tissue. Human RCC tissue samples were obtained and stained for the tumor marker CAIX, as well as CD14, CD16, CD45, CD56, and CD68. Two populations were identified on SSC vs FSC. The populations were further characterized as CAIXhi and CAIX-lo, identifying them and tumor and lymphocytic cells respectively. Both populations were positive for the lymphocytic markers described above. FIG. 2B is CAIX hi tumor cells were gated to identify tumor cells doubly positive for multiple trogocytic markers. The vast majority (~90%) of RCC tumor cells were positive for CD14, CD16, CD56, CD45, and CD68.

FIGS. 3A-3C illustrate evidence of trogocytosis altering gene transcription in renal cell carcinoma (RCC). FIG. 3A demonstrates FACS gating strategy used to isolate lymphocyte (L), trogocytic tumor (T), and non-trogocytic tumor (NT) populations based on tumor marker (CAIX) and lymphocyte marker (CD45) discrimination. FIG. 3B shows representative data of NanoString gene expression analysis performed on human clear cell RCC tumor dissociations. Upper box indicates gene expression characteristic of lymphocyte populations, including new markers of trogocytosis such as CD4 and CD53. The lower box indicates gene expression consistent with RCC tumor cells. FIG. 3C is a plot showing CD45 qRT-PCR gene expression data of various RCC cell lines post coculture with Jurkat T cells relative to un-cocultured RCC controls.

### DETAILED DESCRIPTION OF THE INVENTION

### I. Definitions

The term “antibody” is used in the broadest sense unless clearly indicated otherwise. Therefore, an “antibody” can be naturally occurring or man-made such as monoclonal antibodies produced by conventional hybridoma technology. Antibodies include monoclonal and polyclonal antibodies as well as fragments and polymers containing the antigen-binding domain and/or one or more complementarity determining regions of these antibodies. As used herein, the term “antibody” refers to any form of antibody or antigen binding fragment or recombinant protein, and specifically covers monoclonal antibodies (including full length monoclonal antibodies), polyclonal antibodies, multispecific antibodies (e.g., bispecific antibodies), and antibody fragments so long as they specifically bind the target antigen. Any specific antibody can be used in the methods and compositions provided herein. Thus, in one embodiment the term “antibody” encompasses a molecule comprising at least one variable region from a light chain immunoglobulin molecule and at least one variable region from a heavy chain molecule that in combination form a specific binding site for the target antigen. The term “variable region” is intended to distinguish such domain of the immunoglobulin from domains that are broadly shared by antibodies (such as an antibody Fc domain). The variable region comprises a “hypervariable region” whose residues are responsible for antigen binding. The hypervariable region comprises amino acid residues from a “Complementarity Determining Region” or “CDR” (i.e., typically at approximately residues 24-34 (L1), 50-56 (L2) and 89-97 (L3) in the light chain variable domain and at approximately residues 27-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, MD. (1991)) and/or those residues from a “hypervariable loop” (i.e., residues 26-32 (L1), 50-52 (L2) and 91-96 (L3) in the light chain variable domain and 26-32 (H1), 53-55 (H2) and 96-101 (H3) in the heavy chain variable domain; Chothia, C. et al. (1987) “Canonical Structures For The Hypervariable Regions Of Immunoglobulins,” J. Mol. Biol. 196:901-917). “Framework Region” or “FR” residues are those variable domain residues other than the hypervariable region residues as herein defined. The term antibody includes monoclonal antibodies, multi-specific antibodies, human antibodies, humanized antibodies, synthetic antibodies, chimeric antibodies, camelized antibodies (See e.g., Muyldermans et al., 2001, Trends Biochem. Sci. 26:230; Nuttall et al., 2000, Cur. Pharm. Biotech. 1:253; Reichmann and Muyldermans, 1999, J. Immunol. Meth. 231:25; International Publication Nos. WO 94/04678 and WO 94/25591; U.S. Pat. No. 6,005,079), single-chain Fvs (scFv) (see, e.g., see Pluckthun in The Pharmacology of Monoclonal Antibodies, vol. 113, Rosenburg and Moore eds. Springer-Verlag, New York, pp. 269-315 (1994)), single chain antibodies, disulfide-linked Fvs (sdFv), intrabodies, and anti-idiotypic (anti-Id) antibodies (including, e.g., anti-Id and anti-anti-Id antibodies to antibodies of the invention). In particular, such antibodies include immunoglobulin molecules of any type (e.g., IgG, IgE, IgM, IgD, IgA and IgY), class (e.g., IgG₁, IgG₂, IgG₃, IgG₄, IgA₁ and IgA₂) or subclass. An “antibody fragment” or “antigen binding fragment” of an antibody is defined as at least a portion of the variable region of the immunoglobulin molecule that binds to its target, i.e., the antigen-binding region (also antigen binding domain). In one embodiment it specifically covers single antibodies and clones thereof and anti-antibody compositions with polyepitopic specificity. The antibody of the present methods and compositions can be monoclonal or polyclonal. An antibody can be in the form of an antigen binding antibody fragment including a Fab fragment, F(ab′)₂ fragment, a single chain variable region, and the like. Fragments of intact molecules can be generated using methods well known in the art and include enzymatic digestion and recombinant means. Thus, the “fragment” may be a recombinant protein, e.g., a fusion protein.

As used herein, any form of the “antigen” can be used to generate an antibody that is specific for the target antigen. Thus, the eliciting antigen may be a single epitope, multiple epitopes, or the entire protein alone or in combination with one or more immunogenicity enhancing agents known in the art. The eliciting antigen may be an isolated full-length protein, a cell surface protein (e.g., immunizing with cells transfected with at least a portion of the antigen), or a soluble protein (e.g., immunizing with only the extracellular domain portion of the protein). The antigen may be produced in a genetically modified cell. The DNA encoding the antigen may be genomic or non-genomic (e.g., cDNA) and encodes at least a portion of the extracellular domain. As used herein, the term “portion” refers to the minimal number of amino acids or nucleic acids, as appropriate, to constitute an immunogenic epitope of the antigen of interest. Any genetic vectors suitable for transformation of the cells of interest may be employed, including but not limited to adenoviral vectors, plasmids, and non-viral vectors, such as cationic lipids. In one embodiment, the antibody of the methods and compositions herein specifically bind at least a portion of the extracellular domain of the target antigen of interest.

The antibodies or antigen binding fragments thereof provided herein may be conjugated to a “bioactive agent.” As used herein, the term “bioactive agent” refers to any synthetic or naturally occurring compound that binds the antigen and/or enhances or mediates a desired biological effect.

In one embodiment, the binding fragments useful in the present invention are biologically active fragments. As used herein, the term “biologically active” refers to an antibody or antibody fragment that is capable of binding the desired antigenic epitope and directly or indirectly exerting a biologic effect.

“Bispecific” antibodies are also useful in the present methods and compositions. As used herein, the term “bispecific antibody” refers to an antibody, typically a monoclonal antibody, having binding specificities for at least two different antigenic epitopes. In one embodiment, the epitopes are from the same antigen. In another embodiment, the epitopes are from two different antigens. Methods for making bispecific antibodies are known in the art. For example, bispecific antibodies can be produced recombinantly using the co-expression of two immunoglobulin heavy chain/light chain pairs. See, e.g., Milstein et al., Nature 305:537-39 (1983). Alternatively, bispecific antibodies can be prepared using chemical linkage. See, e.g., Brennan, et al., Science 229:81 (1985). Bispecific antibodies include bispecific antibody fragments. See, e.g., Hollinger, et al., Proc. Natl. Acad. Sci. U.S.A. 90:6444-48 (1993), Gruber, et al., J. Immunol. 152:5368 (1994).

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they specifically bind the target antigen and/or exhibit the desired biological activity (U.S. Pat. No. 4,816,567; and Morrison et al., Proc. Natl. Acad. Sci. USA 81: 6851-6855 (1984)).

The term “specifically binds” or “immuno-specifically binds” refers to the binding of an antibody to its cognate antigen (e.g., CAIX) while not significantly binding to other antigens. Preferably, an antibody “specifically binds” to an antigen with an affinity constant (Ka) greater than about 10⁵ mol⁻¹ (e.g., 10⁶ mol⁻¹, 10⁷ mol⁻¹, 10⁸ mol⁻¹, 10⁹ mol⁻¹, 10¹⁰ mol⁻¹, 10¹¹ mol⁻¹, and 10¹² mol⁻¹ or more) with that second molecule.

The term “monoclonal antibody” or “mAb” refers to an antibody obtained from a substantially homogeneous population of antibodies, i.e., the individual antibodies within the population are identical except for possible naturally occurring mutations that may be present in a small subset of the antibody molecules.

The terms “subject,” “individual,” and “patient” refer to any individual who is the target of treatment using the disclosed compositions. The subject can be a vertebrate, for example, a mammal. Thus, the subject can be a human. The subjects can be symptomatic or asymptomatic. The term does not denote a particular age or sex. A subject can include a control subject or a test subject.

The term “effective amount” or “therapeutically effective amount” means a dosage sufficient to treat, inhibit, or alleviate one or more symptoms of a disease state being treated or to otherwise provide a desired pharmacologic and/or physiologic effect. The precise dosage will vary according to a variety of factors such as subject-dependent variables (e.g., age, immune system health, etc.), the disease, and the treatment being administered. The effect of the effective amount can be relative to a control. Such controls are known in the art and discussed herein, and can be, for example, the condition of the subject prior to or in the absence of administration of the drug, or drug combination.

The term “pharmaceutically acceptable” or “biocompatible” refers to compositions, polymers, and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The phrase “pharmaceutically acceptable carrier” refers to pharmaceutically acceptable materials, compositions, or vehicles, such as a liquid or solid filler, diluent, solvent or encapsulating material involved in carrying or transporting any subject composition, from one organ, or portion of the body, to another organ, or portion of the body. Each carrier must be “acceptable” in the sense of being compatible with the other ingredients of a subject composition and not injurious to the patient.

As used herein, the term “inhibit” means to decrease an activity, response, condition, disease, or other biological parameter. This can include, but is not limited to, the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels. In some forms, the inhibition and reduction are compared according to the level of mRNAs, proteins, cells, tissues, and organs.

The term “treating” or “preventing” a disease, disorder, or condition includes ameliorating at least one symptom of the disease or condition. Desirable effects of treatment include decreasing the rate of disease progression, ameliorating, or palliating the disease state, and remission or improved prognosis. For example, an individual is successfully “treated” if one or more symptoms associated with one or more eating disorders are mitigated or eliminated, including, but are not limited to, decreasing symptoms resulting from the disease, increasing the quality of life of those suffering from the disease, decreasing the dose of other medications required to treat the disease, delaying the progression of the disease, and/or prolonging survival of individuals.

The term “biodegradable”, generally refers to a material that will degrade or erode under physiologic conditions to smaller units or chemical species that are capable of being metabolized, eliminated, or excreted by the subject. The degradation time is a function of composition and morphology.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a ligand is disclosed and discussed and a number of modifications that can be made to a number of molecules including the ligand are discussed, each and every combination and permutation of ligand and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. Further, each of the materials, compositions, components, etc. contemplated and disclosed as above can also be specifically and independently included or excluded from any group, subgroup, list, set, etc. of such materials.

These concepts apply to all aspects of this application including, but not limited to, steps in methods of making and using the disclosed compositions. Thus, if there are a variety of additional steps that can be performed it is understood that each of these additional steps can be performed with any specific form or combination of forms of the disclosed methods, and that each such combination is specifically contemplated and should be considered disclosed.

All methods described herein can be performed in any suitable order unless otherwise indicated or otherwise clearly contradicted by context. The use of any and all examples, or exemplary language (e.g., “such as”) provided herein, is intended merely to better illuminate the forms and does not pose a limitation on the scope of the forms unless otherwise claimed. No language in the specification should be construed as indicating any non-claimed element as essential to the practice of the invention.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other forms the values can range in value either above or below the stated value in a range of approx. +/−5%; in other forms the values can range in value either above or below the stated value in a range of approx. +/−2%; in other forms the values can range in value either above or below the stated value in a range of approx. +/−1%. The preceding ranges are intended to be made clear by context, and no further limitation is implied.

### II. Biomarkers of Trogocytosis and Uses Thereof

Trogocytosis is a process of membrane fragment transfer between two cells in contact (Shin, et al., PNAS, 2021 Vol. 118 No. 48, 9 pages e2110241118). This term “trogo” is derived from ancient Greek meaning “gnaw.” See also, Daubeuf, S., et al., PLoS One, 2010. 5(1): p. e8716, Daubeuf, S., et al., J Immunol, 2010. 184(4): p. 1897-908, Hudrisier, D., et al., J Immunol, 2007. 178(6): p. 3637-47, and Masuda, S., et al., Clin Dev Immunol, 2013. 2013: p. 345745.

It has been established that tumor cells express significant levels of lymphoid proteins, such as, CD56 CD45, CD14, CD16, CD68, CD4 and CD53 via the process of trogocytosis. See also FIG. 3B. Results show that the CD45RA isoform is most commonly found as a trogocytic marker, however CD45RO trogocytic expression occurs, albeit to a lesser extent. Thus, CD45 can be CD45RA or CD45RO. As shown in the Examples, human kidney tumors stably express the lymphoid markers CD45, CD56, CD14, and CD16. FIG. 3C shows CD45 qRT-PCR gene expression data of various renal cell carcinoma (RCC) cell lines increases post coculture with Jurkat T cells relative to un-cocultured RCC controls. This process of membrane-bound proteins being deposited from a donor immune cell to a recipient cancer cell via trogocytosis shapes the immune microenvironment surrounding many types of solid tumors.

Thus, lymphoid proteins (also referred to herein as an immune cell markers), including, but not limited to CD45 (CD45RA and/or CD45RO), CD14, CD16, CD56, CD68, CD4, CD53 and others in FIG. 3B (e.g., PTPRC, FCGR2A, FCGR3A, CTSS) can be used as a biomarkers for cancer diagnosis and/or characterizing cancer cells as being subject to trogocytosis, and identifying the corresponding subject as a target for cancer and/or trogocytosis treatment and guiding such treatment selection. Such compositions and methods are disclosed herein. They can also be used to increase the positive predictive value of current screening modalities and for selection and monitoring the efficacy of treatment regimens.

The methods typically including detecting one or more immune cell markers on non-immune cells of a sample from a subject. In some embodiments, the non-immune cells are suspected or known to be cancer cells. Any of the methods can further include detecting one or more cancer antigen(s), preferably cancer antigen(s) that are not immune cell marker(s). For example, the experiments below show that carbonic anhydrase 9 (CAIX) a commonly used kidney tumor marker, colocalizes with lymphoid markers on kidney cancer cells. In addition to CAIX other common cancer antigens are known in the art and some are expressly provided elsewhere herein and can be detected in the sample as part of the disclosed methods. The presence of both an immune cell marker and non-immune cell marker on non-immune cells can indicate the non-immune cells are cancer cells that have undergone trogocytosis. Thus, these detection methods can be used not only for cancer diagnosis and/or characterization, but also for guiding the selection of cancer treatments as discussed in more detail below.

### A. Source of the Biomarkers

The disclosed biomarkers for cancer and/or trogocytosis are biomolecules, preferably proteins. Some embodiments provide these biomolecules in isolated form. The preferred biological source for detection of the biomarkers is cancer tissue (or tissued suspected of being cancerous) including biopsy material from a tumor. However, in other embodiments, the biomarkers can be isolated from biological fluids. The biomarkers can be isolated by any method known in the art, based on both their mass and their binding characteristics. For example, a sample containing the biomolecules can be subject to, e.g., mass spectroscopy, laser capture microdissection (LCM) and 2D-difference gel electrophoresis, chromatographic fractionation subject to further separation by, e.g., acrylamide gel electrophoresis. Knowledge of the identity of the biomarker also allows their isolation by immunoaffinity chromatography.

### B. Methods of Detecting Biomarkers

The disclosed biomarkers for cancer and/or trogocytosis can be detected by any suitable method. Detection paradigms that can be employed include optical methods, electrochemical methods (voltammetry and amperometry techniques), atomic force microscopy, and radio frequency methods, e.g., multipolar resonance spectroscopy. Illustrative of optical methods, in addition to microscopy, both confocal and non-confocal, are detection of fluorescence, luminescence, chemiluminescence, absorbance, reflectance, transmittance, and birefringence or refractive index (e.g., surface plasmon resonance, ellipsometry, a resonant mirror method, a grating coupler waveguide method or interferometry).

In preferred embodiments, the biomarkers are detected and/or measured by an immunoassay. Immunoassays utilize biospecific capture reagents, such as antibodies, to capture the biomarkers. Antibodies can be produced by methods well known in the art, e.g., by immunizing animals with the biomarkers. Antibodies to immune cell markers such as CD45 (CD45RA and/or CD45RO), CD14, CD16, CD56, CD68 CD4, CD53 and/or others in FIG. 3B are commercially available, and such commercial antibodies are exemplified in the experiments below and can be used in the methods provided herein. Traditional immunoassays including, for example, sandwich immunoassays including ELISA or fluorescence-based immunoassays, as well as other enzyme immunoassays can be used for detecting the biomarkers. In exemplary embodiments, the detection of the biomarker is carried out on slides of test material (e.g., cancer biopsy) or by flow cytometry (FACS analysis), e.g., as exemplified in the experiments below, optionally including the software-based image enhancement activities disclosed therein.

Quantitative immunochemical techniques can also be used. For example, the Quantitative Tissue Biomarker Platform from HistoRx and/or measuring immunofluorescence level(s) can be used to quantify levels of biomarkers.

In some embodiments, a sample is analyzed by means of a biochip. Biochips generally include solid substrates and have a generally planar surface, to which a capture reagent (also called an adsorbent or affinity reagent) is attached. Frequently, the surface of a biochip includes a plurality of addressable locations, each of which has the capture reagent bound there.

Protein biochips are biochips adapted for the capture of polypeptides. Many protein biochips are described in the art. These include; for example, protein biochips produced by Ciphergen Biosystems, Inc. (Fremont, Calif.), Packard BioScience Company (Meriden Conn.), Zyomyx (Hayward, Calif.), Phylos (Lexington, Mass.) and Biacore (Uppsala, Sweden).

In some embodiments, the biomarkers are detected by mass spectrometry, a methodology that employs a mass spectrometer to detect gas phase ions. Examples of mass spectrometers are time-of-flight, magnetic sector, quadrupole filter, ion trap, ion cyclotron resonance, electrostatic sector analyzer, Matrix-Assisted Laser Desorption Ionization-Time-Of-Flight “MALDI-TOF”, Surface Enhanced Laser Description and Ionization or “SELDI,” and hybrids of these.

For example, in some embodiments, the mass spectrometer is a laser desorption/ionization mass spectrometer. In laser desorption/ionization mass spectrometry, the analytes are placed on the surface of a mass spectrometry probe, a device adapted to engage a probe interface of the mass spectrometer and to present an analyte to ionizing energy for ionization and introduction into a mass spectrometer. A laser desorption mass spectrometer employs laser energy, typically from an ultraviolet laser, but also from an infrared laser, to desorb analytes from a surface, to volatilize and ionize them and make them available to the ion optics of the mass spectrometer. Mass spectrometry-based Multi-Reaction Monitoring can also be used to detect the biomarkers. Mass spectrometry-based Multi-Reaction Monitoring has been described by Gerber et al., Proc Natl Acad Sci. USA., 100(12):6940-5 (2003).

### C. Diagnosis

### 1. Single Markers

The biomarkers can be used in diagnostic tests to assess cancer and/or trogocytosis status in a subject, e.g., to distinguish between normal cells and cancer cells, and cancer status. For example, disease status includes, without limitation, the presence or absence of disease (e.g., cancer v. non-cancer), characterization of cells including cancer cells (e.g., trogocytosis v. non-trogocytosis), the risk of developing disease, the stage of the disease (e.g., non-invasive or early-stage cancer v. invasive or metastatic cancer), the progress of disease (e.g., progress of disease or remission of disease over time) and the effectiveness or response to treatment of disease. Based on this status, further procedures may be indicated, including additional diagnostic tests or therapeutic procedures or regimens. Representative cancers and therapies are discussed in more detail below.

The biomarkers discussed herein can be present and/or expressed in cancer including but not limited to kidney cancer, and, therefore, each is individually useful in aiding in the determination of cancer and/or trogocytosis status. The method involves, first, measuring the selected biomarker in a subject sample using the methods described herein, and, second, comparing the measurement with a diagnostic amount or cut-off that distinguishes a positive cancer and/or trogocytosis status from a negative cancer and/or trogocytosis status. The diagnostic amount represents a measured amount of a biomarker above which a subject is classified as having a particular status. For example, if the biomarker is up-regulated compared to normal during cancer, then a measured amount above the diagnostic cutoff provides a diagnosis or status of the cancer. As is well understood in the art, by adjusting the particular diagnostic cut-off used in an assay, one can increase sensitivity or specificity of the diagnostic assay depending on the preference of the diagnostician. The particular diagnostic cut-off can be determined, for example, by measuring the amount of the biomarker in a statistically significant number of samples from subjects with the different cancer statuses and drawing the cut-off to suit the diagnostician's desired levels of specificity and sensitivity.

### 2. Combinations of Markers

While individual biomarkers are useful diagnostic biomarkers, a combination of biomarkers may provide greater predictive value of a particular status than single biomarkers alone. Specifically, the detection of a plurality of biomarkers in a sample can increase the sensitivity and/or specificity of the test. Thus, in one embodiment, two or more, three or more, four or more or even five or more of the biomarkers can be detected and used to assess the status of cancer and/or trogocytosis in a subject.

### D. Determining Risk of Developing Disease

Methods for determining the risk of developing disease in a subject are also provided. Biomarker amounts or patterns can be characteristic of various risk states, e.g., high, medium, or low. The risk of developing a disease is determined by measuring the relevant biomarker or biomarkers and then either submitting them to a classification algorithm or comparing them with a reference amount and/or pattern of biomarkers that is associated with the particular risk level.

### E. Determining Stage of Disease

Another embodiment provides methods for determining the stage of disease in a subject. Each stage of the disease can have a characteristic amount of a biomarker or relative amounts of a set of biomarkers (a pattern). The stage of a disease is determined by measuring the relevant biomarker or biomarkers and then either submitting them to a classification algorithm or comparing them with a reference amount and/or pattern of biomarkers that is associated with the particular stage.

### F. Determining Course (Progression/Remission) of Disease

Still another embodiment provides methods for determining the course of disease in a subject. Disease course refers to changes in disease status over time, including disease progression (worsening) and disease regression (improvement). Over time, the amounts or relative amounts (e.g., the pattern) of the biomarkers changes. This method involves measuring one or more biomarkers in a subject at at least two different time points, e.g., a first time and a second time, and comparing the change in amounts, if any. The course of disease is determined based on these comparisons. Similarly, this method is useful for determining the response to treatment. If a treatment is effective, then the biomarkers will trend toward normal, while if treatment is ineffective, the biomarkers will trend toward disease indications.

### G. Subject Management

In certain embodiments of the method including the detection and/or analysis of one or more biomarkers further include managing subject treatment based on the status. Such management includes the actions of the physician or clinician subsequent to determining cancer and/or trogocytosis status. For example, if a physician makes a diagnosis of cancer and/or trogocytosis, then a certain regime of treatment, such as prescription or administration of chemotherapy, radiation, immunotherapy, including, but not limited to administration of the compositions discussed in more detail below, might follow. Alternatively, a diagnosis of non-cancer or benign tumor might be followed with further testing to determine a specific disease that the patient might be suffering from. Also, if the diagnostic test gives an inconclusive result on cancer and/or trogocytosis status, further tests may be required.

One embodiment provides a method for selecting a subject for treatment for cancer and/or trogocytosis by detecting the presence or quantity of one or more biomarkers provided herein in a sample from a subject suspected of having cancer and/or trogocytosis, comparing the levels of biomarker in the sample to a predetermined standard, wherein the patient is selected for treatment for cancer and/or trogocytosis if certain biomarkers or levels of biomarkers are detected in the sample.

Additional embodiments relate to the communication of assay results or diagnoses or both to technicians, physicians or patients, for example. In certain embodiments, computers will be used to communicate assay results or diagnoses or both to interested parties, e.g.: physicians and their patients. In some embodiments, the assays will be performed or the assay results analyzed in a country or jurisdiction which differs from the country or jurisdiction to which the results or diagnoses are communicated.

In a preferred embodiment a diagnosis based on the presence or absence in a test subject of any of the disclosed biomarkers is communicated to the subject as soon as possible after the diagnosis is obtained. The diagnosis may be communicated to the subject by the subject's treating physician. Alternatively, the diagnosis may be sent to a test subject by email or communicated to the subject by phone. A computer may be used to communicate the diagnosis by email or phone. In certain embodiments, the message containing results of a diagnostic test may be generated and delivered automatically to the subject using a combination of computer hardware and software which will be familiar to artisans skilled in telecommunications. In certain embodiments all or some of the method steps, including the assaying of samples, diagnosing of diseases, and communicating of assay results or diagnoses, may be carried out in diverse (e.g., foreign) jurisdictions.

### H. Biomarkers in Screening Assays

The biomarkers can be used to screen for compounds that modulate the expression of the biomarkers in vitro or in vivo, which compounds in turn may be useful in treating or preventing cancer and/or trogocytosis in patients. Compounds suitable for therapeutic testing may be screened initially by identifying compounds which reduce the presence of one or more biomarkers on the cancer cells.

In a related embodiment, the ability of a test compound to inhibit or otherwise reduce the activity of trogocytosis may be measured. Techniques used to measure the activity of trogocytosis may vary depending on the function and properties of the biomarker. The ability of potentially therapeutic test compounds to inhibit trogocytosis, optionally of the disclosed biomarkers, and can be determined by measuring the biomarkers on cancer cells before and after treatment with the compounds.

Test compounds capable of modulating the presence and/or expression of any of the biomarkers on cancer may be administered to patients who are suffering from or are at risk of developing cancer having the biomarkers. For example, the administration of a test compound which decreases the activity of a particular biomarker may decrease the risk of cancer and/or trogocytosis in a patient if the increased activity of the biomarker is responsible or indicative, at least in part, for the onset of cancer and/or trogocytosis.

At the clinical level, screening a test compound includes obtaining samples from test subjects before and after the subjects have been exposed to a test compound. The levels in the samples of one or more of the biomarkers can be measured and analyzed to determine whether the levels of the biomarkers change after exposure to a test compound. The samples can be analyzed by any appropriate means known to one of skill in the art including e.g., by the means described herein. In a further embodiment, the changes in the level of expression of one or more of the biomarkers can be measured using in vitro methods and materials. For example, human tissue cultured cells which express, or are capable of expressing, one or more of the biomarkers may be contacted with test compounds. Subjects who have been treated with test compounds will be routinely examined for any physiological effects which may result from the treatment. In particular, the test compounds will be evaluated for their ability to decrease disease likelihood in a subject. Alternatively, if the test compounds are administered to subjects who have previously been diagnosed with cancer and/or trogocytosis, test compounds will be screened for their ability to slow or stop the progression of the disease.

I. Assessing the Effectiveness of Treatment or Risk for Developing Cancer and/or Trogocytosis

Methods for determining the course of cancer and/or trogocytosis in a subject are also provided. Disease course refers to changes in disease status over time, including disease progression (worsening) and disease regression (improvement). Over time, the amounts or relative amounts (e.g., the pattern) of the biomarkers changes. Accordingly, this method involves measuring one or more biomarkers in a subject at least two different time points, e.g., a first time and a second time, and comparing the change in amounts, if any. The course of disease is determined based on these comparisons. Similarly, this method is useful for determining the response to treatment. If a treatment is effective, then the biomarkers will trend toward normal, while if treatment is ineffective, the biomarkers will trend toward disease indications.

In yet another example, the biomarkers can be used in heredity studies to determine if the subject is at risk for developing cancer and/or trogocytosis.

### III. Compositions

Compositions for treating or preventing one or more symptoms of cancer in a subject in need thereof are described. For example, compositions that selectively target trogocytosed lymphoid proteins on cancer cells are provided. In some embodiments, the compositions selectively target one or more cancer cells having membrane-bound proteins from a donor immune cell are also described. In preferred embodiments, the compositions include a mono-, bi-, or multispecific antibody selectively targeting tumor cells.

The antibodies typically include an antigen binding site that binds to lymphoid protein (i.e., immune cell maker) present on non-immune cells, more preferably cancer cells. Preferably, immune cell marker is one that is deposited from immune cells onto tumor cell surface. In some embodiments, such antigen binding sites bind to CD45 (CD45RA and/or CD45RO), CD14, CD16, CD56, CD68, CD4, CD53, PTPRC, FCGR2A, FCGR3A, and/or CTSS.

The antibodies can also include an antigen-binding site that selectively targets a conventional cancer antigen (one that expressed higher in cancer cells more than corresponding healthy cells). In this way, in some embodiments, the antibody is a bispecific or multispecific antibody is one that binds to two different antigens on the cancer cells, i.e., an immune cell marker transferred to cancer cell by trogocytosis and a traditional cancer antigen.

Additionally or alternatively the antibodies can include an antigen binding site that binds to an immune cell marker not present on the cancer cells. In this way, in some embodiments, the antibody is bispecific or multispecific antibody that can simultaneously bind to both cancer cells and immune cells.

Thus, in some embodiments, compositions include an antibody or antibody fragment thereof which is a monospecific, bispecific, trispecific, or multispecific antibody. In preferred embodiments, the antibody or antibody fragment thereof is a bispecific antibody. In further preferred embodiments, the antibody or antibody fragment thereof is conjugated to one of more of cytotoxic agents, drugs, receptors, enzymes, receptor ligands.

In some embodiments, the antibody is an antibody-drug conjugate having a cytotoxic payload. In other embodiments, the antibody has an effector function through its Fc region to mediate antibody-dependent cell-mediated cytotoxicity.

Thus, at least two different types of bispecific and multispecific antibodies are provided: (i) antibodies targeting two different antigens present on the cancer cells, wherein at least one of the antigens is an immune cell marker; and (ii) antibodies targeting one tumor antigen present on the cancer cells and one immune-related molecule that is not present on the cancer cells. In some embodiments, the antibodies belonging to the second category is a bispecific T cell engager (BiTE).

Trogocytosis is also involved in attenuating therapeutic efficacy in tumor therapies. Thus, compositions selectively targeting this process to limit the transfer of membrane-bound proteins from a donor immune cell to a recipient cancer cell are also described. In preferred embodiments, compositions include one or more inhibitors of trogocytosis are suitable for enhancing therapeutic efficacy in tumor therapies in a subject.

### A. Antibody-Targeted Therapy

Targeted therapy utilizing monoclonal antibodies (mAbs) has established immunotherapy as a powerful new tool in the fight against cancer. Antibodies directed towards tumor cell antigens can cause tumor cell death by both direct and indirect mechanisms. Direct mechanisms for therapeutic antibodies to induce cell death include blocking growth factor receptor signaling, direct transmembrane signaling, and acting as targeted vectors for toxic payloads such as radioisotopes. In some embodiments, such antibodies are antibody-drug conjugates. The indirect mechanisms require engagement with components of the host immune system and are comprised of complement-mediated cytotoxicity, antibody-dependent cellular phagocytosis, and antibody-dependent cell-mediated cytotoxicity (ADCC).

In some embodiments, the composition includes one or more antibodies selectively targets one or more antigens present on the surface of the cancer cells, preferably with an effector function. In preferred embodiments, the antibodies are associated or conjugated to a toxic payload. In other embodiments, effector function is through the Fc region of the antibodies, for example, modifications in the Fc region results in an antibody with an altered antibody-mediated effector function, an altered binding to other Fc receptors (e.g., Fc activation receptors), an altered antibody-dependent cell-mediated cytotoxicity (ADCC) activity, an altered C1q binding activity, an altered complement-dependent cytotoxicity activity (CDC), a phagocytic activity, or any combination thereof.

In some embodiments, the composition includes one or more antibodies that are modified to alter the half-lives (e.g., serum half-lives) of parental antibodies in a mammal, preferably a human. Preferably such alteration will result in a half-life of greater than 15 days, preferably greater than 20 days, greater than 25 days, greater than 30 days, greater than 35 days, greater than 40 days, greater than 45 days, greater than 2 months, greater than 3 months, greater than 4 months, or greater than 5 months. The increased half-lives of the humanized antibodies of the present invention or fragments thereof in a mammal, preferably a human, results in a higher serum titer of said antibodies or antibody fragments in the mammal, and thus, reduces the frequency of the administration of said antibodies or antibody fragments and/or reduces the concentration of said antibodies or antibody fragments to be administered. Antibodies or fragments thereof having increased in vivo half-lives can be generated by techniques known to those of skill in the art. For example, antibodies or fragments thereof with increased in vivo half-lives can be generated by modifying (e.g., substituting, deleting, or adding) amino acid residues identified as involved in the interaction between the Fc domain and the FcRn receptor. The humanized antibodies of the invention may be engineered to increase biological half-lives (see, e.g., U.S. Pat. No. 6,277,375). For example, humanized antibodies of the invention may be engineered in the Fc-hinge domain to have increased in vivo or serum half-lives.

Antibodies or fragments thereof with increased in vivo half-lives can be generated by attaching to said antibodies or antibody fragments polymer molecules such as high molecular weight polyethyleneglycol (PEG). PEG can be attached to said antibodies or antibody fragments with or without a multifunctional linker either through site-specific conjugation of the PEG to the N- or C-terminus of said antibodies or antibody fragments or via epsilon-amino groups present on lysine residues. Linear or branched polymer derivatization that results in minimal loss of biological activity will be used. The degree of conjugation will be closely monitored by SDS-PAGE and mass spectrometry to ensure proper conjugation of PEG molecules to the antibodies. Unreacted PEG can be separated from antibody-PEG conjugates by, e.g., size exclusion or ion-exchange chromatography.

### 1. Bispecific Antibodies

In some embodiments, the composition includes a bispecific or multispecific antibody targeting tumor cells by immuno-specifically binding to two or more distinct cell surface molecules present on the tumor cells. In preferred embodiments, the bispecific antibody selectively targets tumor cells with a first antigen-binding site selectively targeting a conventional cancer antigen (one that expressed higher in cancer cells more than corresponding healthy cells) and a second antigen-binding site selectively targeting an immune cells marker, preferably, one deposited from immune cells onto tumor cell surface.

In some embodiments, the composition includes a bispecific or multispecific antibody targeting tumor cells by immuno-specifically binding to one immune cell surface marker present on the tumor cells and one immune cell surface marker present on immune cells, but absence from the tumor cells.

In some embodiments, the antibodies for targeting cancer cells are humanized to reduce immunogenicity in the subject to be treated. In some embodiments, the antibodies are antibodies or fragments thereof that include a CDR(s) that is at least 45%, at least 50%, at least 55%, at least 60%, at least 65%, at least 70%, at least 75%, at least 80%, at least 85%, at least 90%, at least 95%, at least 99%, or at least 100% identical to the amino acid sequence of one, two, three, four, five, or all six CDRs of a known antibody, including but not limited to clone and commercially available antibodies mentioned herein, and which exhibit immuno-specific binding to the intended target. The determination of percent identity of two amino acid sequences can be determined by sequence alignment between to the two proteins.

a. Immune Cell Markers on Tumor Cells

In some embodiments, the disclosed bispecific antibody has a binding specificity towards one or more immune cell surface molecules or immune cell markers. In preferred embodiments, the one or more immune cell markers are those trogocytosed from immune cells onto tumor cells, and more preferably presented on the cell surface of the tumor cells. In other embodiments, the one or more immune cell markers are derived from macrophages, dendritic cells, monocytes, NK cells, and T cells.

Exemplary immune cell surface molecules suitable for use include, but are not limited to, CD45, CD56, CD16, CD14, CD68, CD4, CD53, PTPRC, FCGR2A, FCGR3A, and CTSS. Results show that the CD45RA isoform is most commonly found as a trogocytic marker, however CD45RO trogocytic expression occurs, albeit to a lesser extent. Thus, in some embodiments, CD45 is CD45RA or CD45RO. In other embodiments, immune cell markers are CTLA4, PDL1, Tim3, VISTA, LAG3, CD38, CD80, CD86, MHC Class II, and/or PD-L1.

Antibodies that bind to the foregoing markers are known in the art and commercially available. See also the experiments provided below.

b. Cancer Cell Antigens

In some embodiments, the disclosed bispecific antibody immuno-specifically binds one or more cancer antigens. The cancer antigen can be one that elevated on cancer cells relative to non-cancer cells. The cancer antigen can also be one that present on cancer cells but absent from non-cancer cells.

Exemplary cancer cell antigens suitable for use include, but are not limited to, 4-1BB, 5T4, adenocarcinoma antigen, alpha-fetoprotein, BAFF, BCMA, B-lymphoma cell, C242 antigen, Cancer Antigen 125 (CA-125), carbonic anhydrase 9 (CA-IX), C-MET, CCR4, CD152, CD19, CD20, CD200, CD22, CD221, CD23 (IgE receptor), CD28, CD30 (TNFRSF8), CD33, CD4, CD40, CD44 v6, CD51, CD52, CD56, CD74, CD79b, CD80, CEA, CNT0888, CTLA-4, DR5, EGFR, EpCAM, CD3, FAP, fibronectin extra domain-B, folate receptor 1, GD2, GD3 ganglioside, glycoprotein 75, glycoprotein non-metastatic B (GPNMB), HER2/neu, HGF, human scatter factor receptor kinase, IGF-1 receptor, IGF-I, IgGl, Ll-CAM, IL-13, IL-6, insulin-like growth factor I receptor, integrin α5β1, integrin ανβ3, MORAb-009, MS4A1, MUC1, mucin CanAg, N-glycolylneuraminic acid, Nectin4, NPC-1C, PDGF-R a, PDL192, phosphatidylserine, prostatic carcinoma cells, RANKL, RON, ROR1, SCH 900105, SDC1, SLAMF7, TAG-72, tenascin C, TGF beta 2, TGF-β, TRAIL-R1, TRAIL-R2, trophoblast cell surface antigen (TROP-2), tumor antigen CTAA16.88, VEGF-A, VEGFR-1, VEGFR2, vimentin, and combinations thereof.

Tumor-associated antigens may include, for example, cellular oncogene-encoded products or aberrantly expressed proto-oncogene-encoded products (e.g., products encoded by the neu, ras, trk, and kit genes), or mutated forms of growth factor receptor or receptor-like cell surface molecules (e.g., surface receptor encoded by the c-erb B gene). Other tumor-associated antigens include molecules that may be directly involved in transformation events, or molecules that may not be directly involved in oncogenic transformation events but are expressed by tumor cells (e.g., carcinoembryonic antigen, CA-125, melanoma associated antigens, etc.) (see, e.g., U.S. Pat. No. 6,699,475; Jager, et al., Int. J. Cancer, 106:817-20 (2003); Kennedy, et al., Int. Rev. Immunol., 22:141-72 (2003); Scanlan, et al. Cancer Immun., 4:1 (2004)).

Genes that encode cellular tumor associated antigens include cellular oncogenes and proto-oncogenes that are aberrantly expressed. In general, cellular oncogenes encode products that are directly relevant to the transformation of the cell, and because of this, these antigens are particularly preferred targets for immunotherapy. An example is the tumorigenic neu gene that encodes a cell surface molecule involved in oncogenic transformation. Other examples include the ras, kit, and trk genes. The products of proto-oncogenes (the normal genes which are mutated to form oncogenes) may be aberrantly expressed (e.g., overexpressed), and this aberrant expression can be related to cellular transformation. Thus, the product encoded by proto-oncogenes can be targeted. Some oncogenes encode growth factor receptor molecules or growth factor receptor-like molecules that are expressed on the tumor cell surface. An example is the cell surface receptor encoded by the c-erbB gene. Other tumor-associated antigens may or may not be directly involved in malignant transformation. These antigens, however, are expressed by certain tumor cells and may therefore provide effective targets. Some examples are carcinoembryonic antigen (CEA), CA 125 (associated with ovarian carcinoma), and melanoma specific antigens.

In ovarian and other carcinomas, for example, tumor associated antigens are detectable in samples of readily obtained biological fluids such as serum or mucosal secretions. One such marker is CA125, a carcinoma associated antigen that is also shed into the bloodstream, where it is detectable in serum (e.g., Bast, et al., N. Eng. J. Med., 309:883 (1983); Lloyd, et al., Int. J. Canc., 71:842 (1997). CA125 levels in serum and other biological fluids have been measured along with levels of other markers, for example, carcinoembryonic antigen (CEA), squamous cell carcinoma antigen (SCC), tissue polypeptide specific antigen (TPS), sialyl TN mucin (STN), and placental alkaline phosphatase (PLAP), in efforts to provide diagnostic and/or prognostic profiles of ovarian and other carcinomas (e.g., Sarandakou, et al., Acta Oncol., 36:755 (1997); Sarandakou, et al., Eur. J. Gynaecol. Oncol., 19:73 (1998); Meier, et al., Anticancer Res., 17(4B):2945 (1997); Kudoh, et al., Gynecol. Obstet. Invest., 47:52 (1999)). Elevated serum CA125 may also accompany neuroblastoma (e.g., Hirokawa, et al., Surg. Today, 28:349 (1998), while elevated CEA and SCC, among others, may accompany colorectal cancer (Gebauer, et al., Anticancer Res., 17(4B):2939 (1997)).

The tumor associated antigen, mesothelin, defined by reactivity with monoclonal antibody K-1, is present on a majority of squamous cell carcinomas including epithelial ovarian, cervical, and esophageal tumors, and on mesotheliomas (Chang, et al., Cancer Res., 52:181 (1992); Chang, et al., Int. J. Cancer, 50:373 (1992); Chang, et al., Int. J. Cancer, 51:548 (1992); Chang, et al., Proc. Natl. Acad. Sci. USA, 93:136 (1996); Chowdhury, et al., Proc. Natl. Acad. Sci. USA, 95:669 (1998)). Using MAb K-1, mesothelin is detectable only as a cell-associated tumor marker and has not been found in soluble form in serum from ovarian cancer patients, or in medium conditioned by OVCAR-3 cells (Chang, et al., Int. J. Cancer, 50:373 (1992)). Structurally related human mesothelin polypeptides, however, also include tumor-associated antigen polypeptides such as the distinct mesothelin related antigen (MRA) polypeptide, which is detectable as a naturally occurring soluble antigen in biological fluids from patients having malignancies (see WO 00/50900).

A tumor antigen may include a cell surface molecule. Tumor antigens of known structure and having a known or described function, include the following cell surface receptors: HER1 (GenBank Accession No. U48722), HER2 (Yoshino, et al., J. Immunol., 152:2393 (1994); Disis, et al., Canc. Res., 54:16 (1994); GenBank Acc. Nos. X03363 and M17730), HER3 (GenBank Acc. Nos. U29339 and M34309), HER4 (Plowman, et al., Nature, 366:473 (1993); GenBank Acc. Nos. L07868 and T64105), epidermal growth factor receptor (EGFR) (GenBank Acc. Nos. U48722, and K03193), vascular endothelial cell growth factor (GenBank No. M32977), vascular endothelial cell growth factor receptor (GenBank Acc. Nos. AF022375, 1680143, U48801 and X62568), insulin-like growth factor-I (GenBank Acc. Nos. X00173, X56774, X56773, X06043, European Patent No. GB 2241703), insulin-like growth factor-II (GenBank Acc. Nos. X03562, X00910, M17863 and M17862), transferrin receptor (Trowbridge and Omary, Proc. Nat. Acad. USA, 78:3039 (1981); GenBank Acc. Nos. X01060 and M11507), estrogen receptor (GenBank Acc. Nos. M38651, X03635, X99101, U47678 and M12674), progesterone receptor (GenBank Acc. Nos. X51730, X69068 and M15716), follicle stimulating hormone receptor (FSH-R) (GenBank Acc. Nos. Z34260 and M65085), retinoic acid receptor (GenBank Acc. Nos. L12060, M60909, X77664, X57280, X07282 and X06538), MUC-1 (Barnes, et al., Proc. Nat. Acad. Sci. USA, 86:7159 (1989); GenBank Acc. Nos. M65132 and M64928) NY-ESO-1 (GenBank Acc. Nos. AJ003149 and U87459), NA 17-A (PCT Publication No. WO 96/40039), Melan-A/MART-1 (Kawakami, et al., Proc. Nat. Acad. Sci. USA, 91:3515 (1994); GenBank Acc. Nos. U06654 and U06452), tyrosinase (Topalian, et al., Proc. Nat. Acad. Sci. USA, 91:9461 (1994); GenBank Acc. No. M26729; Weber, et al., J. Clin. Invest, 102:1258 (1998)), Gp-100 (Kawakami, et al., Proc. Nat. Acad Sci. USA, 91:3515 (1994); GenBank Acc. No. S73003, Adema, et al., J. Biol. Chem., 269:20126 (1994)), MAGE (van den Bruggen, et al., Science, 254:1643 (1991)); GenBank Acc. Nos. U93163, AF064589, U66083, D32077, D32076, D32075, U10694, U10693, U10691, U10690, U10689, U10688, U10687, U10686, U10685, L18877, U10340, U10339, L18920, U03735 and M77481), BAGE (GenBank Acc. No. U19180; U.S. Pat. Nos. 5,683,886 and 5,571,711), GAGE (GenBank Acc. Nos. AF055475, AF055474, AF055473, U19147, U19146, U19145, U19144, U19143 and U19142), any of the CTA class of receptors including in particular HOM-MEL-40 antigen encoded by the SSX2 gene (GenBank Acc. Nos. X86175, U90842, U90841 and X86174), carcinoembryonic antigen (CEA, Gold and Freedman, J. Exp. Med., 121:439 (1985); GenBank Acc. Nos. M59710, M59255 and M29540), and PyLT (GenBank Acc. Nos. J02289 and J02038); p97 (melanotransferrin) (Brown, et al., J. Immunol., 127:539-46 (1981); Rose, et al., Proc. Natl. Acad. Sci. USA, 83:1261-61 (1986)).

Additional tumor associated antigens include prostate surface antigen (PSA) (U.S. Pat. Nos. 6,677,157; 6,673,545); β-human chorionic gonadotropin β-HCG) (McManus, et al., Cancer Res., 36:3476-81 (1976); Yoshimura, et al., Cancer, 73:2745-52 (1994); Yamaguchi, et al., Br. J. Cancer, 60:382-84 (1989): Alfthan, et al., Cancer Res., 52:4628-33 (1992)); glycosyltransferase β-1,4-N-acetylgalactosaminyltransferases (GalNAc) (Hoon, et al., Int. J. Cancer, 43:857-62 (1989); Ando, et al., Int. J. Cancer, 40:12-17 (1987); Tsuchida, et al., J. Natl. Cancer, 78:45-54 (1987); Tsuchida, et al., J. Natl. Cancer, 78:55-60 (1987)); NUC18 (Lehmann, et al., Proc. Natl. Acad. Sci. USA, 86:9891-95 (1989); Lehmann, et al., Cancer Res., 47:841-45 (1987)); melanoma antigen gp75 (Vijayasardahi, et al., J. Exp. Med., 171:1375-80 (1990); GenBank Accession No. X51455); human cytokeratin 8; high molecular weight melanoma antigen (Natali, et al., Cancer, 59:55-63 (1987); keratin 19 (Datta, et al., J. Clin. Oncol., 12:475-82 (1994)).

Tumor antigens of interest include antigens regarded in the art as “cancer/testis” (CT) antigens that are immunogenic in subjects having a malignant condition (Scanlan, et al., Cancer Immun., 4:1 (2004)). CT antigens include at least 19 different families of antigens that contain one or more members and that are capable of inducing an immune response, including but not limited to MAGEA (CT1); BAGE (CT2); MAGEB (CT3); GAGE (CT4); SSX (CT5); NY-ESO-1 (CT6); MAGEC (CT7); SYCP1 (C8); SPANXB1 (CT11.2); NA88 (CT18); CTAGE (CT21); SPA17 (CT22); OY-TES-1 (CT23); CAGE (CT26); HOM-TES-85 (CT28); HCA661 (CT30); NY-SAR-35 (CT38); FATE (CT43); and TPTE (CT44).

Additional tumor antigens that can be targeted, including a tumor-associated or tumor-specific antigen, include, but not limited to, alpha-actinin-4, Bcr-Abl fusion protein, Casp-8, beta-catenin, cdc27, cdk4, cdkn2a, coa-1, dek-can fusion protein, EF2, ETV6-AML1 fusion protein, LDLR-fucosyltransferase AS fusion protein, HLA-A2, HLA-A11, hsp70-2, KIAAO205, Mart2, Mum-1, 2, and 3, neo-PAP, myosin class I, OS-9, pml-RARα fusion protein, PTPRK, K-ras, N-ras, Triosephosphate isomeras, Bage-1, Gage 3, 4, 5, 6, 7, GnTV, Herv-K-mel, Lage-1, Mage-A1, 2, 3, 4, 6, 10, 12, Mage-C2, NA-88, NY-Eso-1/Lage-2, SP17, SSX-2, and TRP2-Int2, MelanA (MART-I), gp100 (Pmel 17), tyrosinase, TRP-1, TRP-2, MAGE-1, MAGE-3, BAGE, GAGE-1, GAGE-2, p15(58), CEA, RAGE, NY-ESO (LAGE), SCP-1, Hom/Mel-40, PRAME, p53, H-Ras, HER-2/neu, BCR-ABL, E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2, p180erbB-3, c-met, nm-23H1, PSA, TAG-72-4, CA 19-9, CA 72-4, CAM 17.1, NuMa, K-ras, β-Catenin, CDK4, Mum-1, p16, TAGE, PSMA, PSCA, CT7, telomerase, 43-9F, 5T4, 791Tgp72, α-fetoprotein, 13HCG, BCA225, BTAA, CA 125, CA 15-3 (CA 27.29\BCAA), CA 195, CA 242, CA-50, CAM43, CD68\KP1, CO-029, FGF-5, G250, Ga733 (EpCAM), HTgp-175, M344, MA-50, MG7-Ag, MOV18, NB\70K, NY-CO-1, RCAS1, SDCCAG16, TA-90 (Mac-2 binding protein\cyclophilin C-associated protein), TAAL6, TAG72, TLP, and TPS. Other tumor-associated and tumor-specific antigens are known to those of skill in the art and are suitable for targeting by the disclosed fusion proteins.

In some embodiments, the bispecific antibody has at least one of the binding sites derived from existing cancer-specific antibodies known in the art. Exemplary cancer-specific antibodies include therapeutic hmAbs. Exemplary therapeutic hmAbs include FDA approved therapeutic monoclonal antibodies which include, but are not limited to, ACTEMRA® (tocilizumab, GENENTECH), ADCETRIS® (brentuximab vedotin, SEATTLE GENETICS), AMJEVITA® (adalimumab-atto, AMGEN INC), ANTHIM® (obiltoxaximab, ELUSYS THERAPEUTICS INC), ARZERRA® (ofatumumab, GLAXO GRP LTD), AVASTIN® (bevacizumab, GENENTECH), BAVENCIO® (avelumab, EMD SERONO INC), BENLYSTA® (belimumab, HUMAN GENOME SCIENCES INC.), BESPONSA® (inotuzumab ozogamicin, WYETH PHARMS INC), BLINCYTO® (blinatumomab, AMGEN), CAMPATH® (alemtuzumab, GENZYME), CIMZIA® (certolizumab pegol, UCB INC), CINQAIR® (reslizumab, TEVA RESPIRATORY LLC), COSENTYX® (secukinumab, NOVARTIS PHARMS CORP), CYLTEZO® (adalimumab-adbm, BOEHRINGER INGELHEIM), CYRAMZA® (ramucirumab, ELI LILLY AND CO), DARZALEX® (daratumumab, JANSSEN), DERMABET® (betamethasone valerate, TARO), DUPIXENT® (dupilumab, REGENERON PHARMACEUTICALS), EMPLICITI® (elotuzumab, BRISTOL MYERS SQUIBB), ENTYVIO® (vedolizumab, TAKEDA PHARMS USA), ERBITUX® (cetuximab, IMCLONE), FASENRA® (benralizumab, ASTRAZENECA AB), GAZYVA® (obinutuzumab, GENENTECH), HEMLIBRA® (emicizumab, GENENTECH INC), HERCEPTIN® (trastuzumab, GENENTECH), HUMIRA® (adalimumab, ABBVIE INC), ILARIS® (canakinumab, NOVARTIS PHARMS), ILUMYA® (tildrakizumab-asmn, MERCK SHARP DOHME), IMFINZI® (durvalumab, ASTRAZENECA UK LTD), INFLECTRA® (infliximab-dyyb, CELLTRION INC), IXIFI® (infliximab-qbtx, PFIZER INC), KADCYLA® (ado-trastuzumab emtansine, GENENTECH), KEVZARA® (sarilumab, SANOFI SYNTHELABO), KEYTRUDA® (pembrolizumab, MERCK SHARP DOHME), LARTRUVO® (olaratumab, ELI LILLY AND CO), LEMTRADA® (alemtuzumab, GENZYME), LUCENTIS® (ranibizumab, GENENTECH), MVASI® (bevacizumab-awwb, AMGEN INC), MYLOTARG® (gemtuzumab ozogamicin, WYETH PHARMS INC), MYOSCINT® (imciromab pentetate, CENTOCOR INC), NUCALA® (mepolizumab, GLAXOSMITHKLINE LLC), OCREVUS® (ocrelizumab, GENENTECH INC), OGIVRI® (trastuzumab-dkst, MYLAN GMBH), OPDIVO® (nivolumab, BRISTOL MYERS SQUIBB), PERJETA® (pertuzumab, GENENTECH), PORTRAZZA® (necitumumab, ELI LILLY CO), PRALUENT® (alirocumab, SANOFI AVENTIS), PRAXBIND® (idarucizumab, BOEHRINGER INGELHEIM), PROLIA® (denosumab, AMGEN), PROSTASCINT® (capromab pendetide, CYTOGEN), RAXIBACUMAB® (raxibacumab, HUMAN GENOME SCIENCES INC.), REMICADE® (infliximab, CENTOCOR INC), RENFLEXIS® (infliximab-abda, SAMSUNG BIOEPSIS CO LTD), REOPRO® (abciximab, CENTOCOR INC), REPATHA® (evolocumab, AMGEN INC), RITUXAN® (rituximab, GENENTECH), SILIQ® (brodalumab, VALEANT LUXEMBOURG), SIMPONI ARIA® (golimumab, JANSSEN BIOTECH), SIMULECT® (basiliximab, NOVARTIS), SOLIRIS® (eculizumab, ALEXION PHARM), STELARA® (ustekinumab, CENTOCOR ORTHO BIOTECH INC), STELARA® (ustekinumab, JANSSEN BIOTECH), SYLVANT® (siltuximab, JANSSEN BIOTECH), SYNAGIS® (palivizumab, MEDIMMUNE), TALTZ® (ixekizumab, ELI LILLY AND CO), TECENTRIQ® (atezolizumab, GENENTECH INC), TREMFYA® (guselkumab, JANSSEN BIOTECH), TROGARZO® (ibalizumab-uiyk, TAIMED BIOLOGICS USA), TYSABRI® (natalizumab, BIOGEN IDEC), UNITUXIN® (dinutuximab, UNITED THERAP), VECTIBIX® (panitumumab, AMGEN), XGEVA® (denosumab, AMGEN), XOLAIR® (omalizumab, GENENTECH), YERVOY® (ipilimumab, BRISTOL MYERS SQUIBB), ZEVALIN® (ibritumomab tiuxetan, SPECTRUM PHARMS), ZINBRYTA® (daclizumab, BIOGEN), ZINPLAVA® (bezlotoxumab, MERCK SHARP DOHME). In some embodiments, antibody is or includes an anti-CAIX antibody, or an anti-HER2 antibody.

c. Immune Cell Makers Absent from Tumor Cells

In some embodiments, the antibody binds to an immune cell marker present on cancer cells such as, but not limited to, CD45 (CD45RA and/or CD45RO), CD14, CD16, CD56, CD68 CD4, CD53 and others in FIG. 3B (e.g., PTPRC, FCGR2A, FCGR3A, and CTSS). and a second immune cell marker preferably one that is highly expressed on immune cells and/or low or absent on the cancer cells. Such antibodies can be used to bring and/or maintain immune cells in close proximity to cancer cells so an immune response can be carried out against the cancer cells.

In some embodiments, the second immune cell marker is B220/CD45R, CCR10, CCR127, CD2, CD3, CD4, CD5, CD8, CD11a, CD19, CD20, CD25, CD27, CD28, CD31, CD34, CD38, CD45RA, CD45RO, CD56, CD58, CD62L, CD103, CD122, CD161, CD194, CD195, CD196, CD197, CD294, Fc gamma RIII (CD16), Fc gamma RIIIA/CD16a, Fc gamma RIIIB/CD16b, Flt-3/Flk-2, IL-7R alpha/CD127, NCAM-1/CD56, Neprilysin/CD10, NKp46/NCR1, or Siglec-2/CD22.

In some embodiments, the second immune cell marker is selected from Table 1.

[symbol]

e 1
Human Immune Cell Markers

| [symbol] type | Markers |
|---|---|
| [symbol] matopoietic stem cells (HSC) | CD34+, CD38−, CD45RA−, CD49+, CD90/Thy1+ |
| [symbol] ulti-potent progenitor (MPP) | CD34+, CD38−, CD45RA−, CD90/Thy1− |
| Common lymphoid progenitor (CLP) | CD34+, CD38+, CD10+, CD45RA+ |
| Common myeloid progenitor (CMP) | CD34+, CD38+, CD7−, CD10−, CD45RA−, CD90/Thy1−, CD135+ |
| Megakaryocyte-erythroid progenitor (MEP) | CD34+, CD38+, CD7−, CD10−, CD45RA−, CD135−, IL3Rα− |
| Granulocyte-monocyte progenitor (GMP) | CD34+, CD38+, CD10−, CD45RA+, CD123+, CD135+ |
| Natural killer cell | CD3−, CD56+, CD94+, NKp46+ |
| T-cell | CD3+ |
| B-cell | CD19+ |
| Monocyte | CD14+ |
| Macrophage | CD11b+, CD68+, CD163+ |
| Dendritic cell | CD11c+, HLA-DR+ |
| Neutrophil | CD11b+, CD16+, CD18+, CD32+, CD44+, CD55+ |
| Eosinophil | CD45+, CD125+, CD193+, F4/80+, Siglec-8+ |
| Basophil | CD19−, CD22+, CD45low, CD123+ |
| Mast cell | CD32+, CD33+, CD117+, CD203c+, FcεRI+ |
| Erythrocyte | CD235a+ |
| Megakaryocyte | CD41b+, CD42a+, CD42b+, CD61+ |

[symbol]

indicates data missing or illegible when filed

macrophages, with some evidence indicating that CD68 is also found on fibroblasts. CD16 is an Fc receptor expressed on naïve, CD56dim NK cells prior to activation. CD56 is the archetypal phenotypic marker of NK cells, but it can also be expressed by alpha beta T cells, gamma delta T cells, dendritic cells, and monocytes. Finally, CD45 expression occurs on all nucleated hematopoietic cells. CD45 has several isoforms that occur due to alternative splicing, with CD45RO being expressed on memory T cells, and CD45RA being expressed on naïve CD4+ and CD8+ T cells.

Antibodies that bind to the foregoing markers are known in the art and commercially available.

In some embodiments, the antibody is a bispecific T cell engager (BiTE). In some embodiments, the BiTE targets an immune marker on cancer cells and CD3 and/or CD19. In some embodiments, the BiTE is an IgG-based antibody or a variable fragment (Fv)-based antibody. Antibody technology used for preparing BiTE can be used in the disclosed compositions and methods, and include, but are not limited to, those described in Tian, et al., “Bispecific T cell engagers: an emerging therapy for management of hematologic malignancies”, J Hematol Oncol 14, 75 (2021). doi.org/10.1186/s13045-021-01084-4, which is specifically incorporated by reference herein in its entirety.

Preferably the immune cell marker absent from the cancer cells.

### 2. Antibody-Drug Conjugate

Antibody-drug conjugates (ADCs) are antibodies, or more typically antibody fragments, that bind surface proteins expressed specifically or preferentially on target cells (e.g., cancer cells), are internalized and then release their cytotoxic agent killing the target cells. There are three components to an ADC: the antibody or antibody fragment (jointly referred to herein as “antibody”), the cytotoxic agent and a chemical linker that connects them. The antibody needs to bind to a surface epitope that will target the ADC to cancer cells. The linker needs to keep the cytotoxic agent attached to the antibody until internalization, and then release the cytotoxic agent. The cytotoxic agent needs to be exceptionally potent since this system releases a limited amount of the drug inside cancer cells. Antibodies, cytotoxic agents, linkers, and methods for conjugation are described, for example, in U.S. Pat. No. 8,871,908.

Due to the low oral bioavailability of ADCs, ADCs are typically administered by intravenous injection. ADCs circulating in the blood bind their target cells. After binding, the ADC-antigen complex is internalized by clathrin-mediated endocytosis to form an early endosome containing an ADC-antigen complex. The early endosome eventually develops into a secondary endosome prior to fusion with the lysosome. For ADCs with cleavable linkers, the cleavage mechanism (e.g., hydrolysis, protease cleavage, disulfide bond cleavage) may occur either in the early endosome or in the secondary endosome, but not in lysosomal transport phase. However, for ADCs with non-cleavable linkers, the release of cytotoxic agents (drugs) is achieved by complete protein degradation in lysosomes: proton pumps in lysosomes create an acidic environment that promotes protease (e.g., cathepsin-B, plasmin) mediated proteolytic cleavage.

The desirable properties of the ADC antibody portion include: 1) minimal immunogenicity; 2) high affinity and avidity for tumor antigen, and efficient internalization (ADC-target antigen complexes need to be internalized by receptor-mediated endocytosis, allowing them to release potent cytotoxic loads in cells); 3) longer circulating half-life.

In terms of specificity, an ideal target antigen needs to have two characteristics at the same time: 1) high expression on the surface of target cells; and 2) low expression in healthy tissues. In addition, the ideal shedding of the antibody should be as small as possible to prevent the free antigen from binding to the antibody in the circulation.

Typically, the antibody suitable for delivery of the active agents is a bispecific antibody described above, having one antigen-binding site selectively targeting a conventional cancer antigen and another antigen-binding site selectively targeting an immune cells marker.

a. Cytotoxic Payloads

The cytotoxic payload is the effector component of the ADC. In some embodiments, the cytotoxic agent of the ADC can target either DNA or tubulin. Cytotoxic agents include anti-microtubules agents (e.g., maytansinoids or auristatins) and DNA-damaging agents (e.g., calicheamicins). Auristatins, like monomethyl auristatin E (MMAE) and F (MMAF), are synthetic compounds derived from dolastatin 10, a natural antimitotic drug. Brentuximab vedotin, polatuzumab vedotin and enfortumab vedotin are all approved ADCs carrying the MMAE payload. Maytansinoids (DM1 and DM4) are synthetic derivatives of maytansine that also act by inhibiting microtubule polymerization. DM1 is the warhead carried by trastuzumab emtansine (T-DM1), the first ADC approved for solid tumors.

The effect of tubulin inhibitors DM1, DM4, MMAE (auristatins monomethyl auristatin E) and MMAF (monomethyl auristatin F) is to inhibit microtubule polymerization, resulting in G2/M phase cell cycle arrest.

The basic parameters for selecting the cytotoxic agent include conjugation, solubility, and stability. The structure of the cytotoxic agent should be such that it can be coupled to a linker. In addition, the water solubility of the toxic molecule and the long-term stability in the blood are important because the ADCs are prepared in an aqueous solution and administered intravenously.

The linker binds the cytotoxic agent to the mAb and maintains ADC stability in the systemic circulation. The chemical nature of the linker and the conjugating site play a crucial role in the stability, pharmacokinetic and pharmacodynamic properties of the ADC, as well as the therapeutic window. In some embodiments, the linker includes peptides and/or PEG chain. In preferred embodiments, the linker is designed to mitigate aggregation and immunogenicity of the ADC.

An ideal linker must have sufficient stability to ensure that the ADC molecules do not break apart early, can safely circulate through the bloodstream, and reach the target site. The linker must be able to break quickly during internalization to release the cytoxic agent. Linkers are classified into two types based on mechanism of cleavage: cleavable and non-cleavable. The former relies on physiological environment to release cytotoxic agents. A non-cleavable linker is a non-reducible bond with an amino acid residue in an mAb and is, therefore, more stable in the blood. An example of such a linker is a thioether linker, dependent on the lysosomal degradation of the mAb to release its cytotoxic agent.

The conjugating characteristics of the connector are critical to control the therapeutic window of the ADC. The drug to antibody ratio (DAR), or the amount of cytotoxic agent attached to the mAb, determines the potency and toxicity of the ADC. Although high drug loading can increase the potency of the ADC, it also increases off-target effects. To overcome the variability in the DARs of the ADC drugs in the production process, some studies have adopted site-specific conjugation to reduce variability, improve conjugating stability, and pharmacokinetic properties, and ultimately provide higher yield of the ADC drugs with a desired DAR. In some embodiments, the drug to antibody ratio (DAR) is between about 1:1 to about 20:1, inclusive; preferably between about 1:1 to about 10:1, inclusive; or between about 2:1 to about 5:1, inclusive.

Table 2 lists FDA approved ADCs.

TABLE 2
FDA approved ADCs

| | | | | Cytotoxic | |
|---|---|---|---|---|---|
| | | | | agent/Cytotoxic | Cytotoxic |
| ADC | Target | mAb | Linker | agent Class | agent Action |
| MYLOTARG ® | CD33 | IgG4 | acid | ozogamicin/ | DNA cleavage |
| (gemtuzumab | | | cleavable | calicheamicin | |
| ozogamicin) | | | | | |
| ADCETRIS ® | CD30 | IgG1 | enzyme | MMAE/ | microtubule |
| (brentuximab vedotin) | | | cleavable | auristatin | inhibitor |
| KADCYLA ® | HER2 | IgG1 | non- | DM1/ | microtubule |
| (adotrastuzumab | | | cleavable | maytansinoid | inhibitor |
| emtansine) | | | | | |
| BESPONSA ® | CD22 | IgG4 | acid | ozogamicin/ | DNA cleavage |
| (inotuzumab | | | cleavable | calicheamicin | |
| ozogamicin) | | | | | |
| POLIVY ® | CD79b | IgG1 | enzyme | MMAE | microtubule |
| (polatuzumab | | | cleavable | auristatin | inhibitor |
| vedotin-piiq) | | | | | |
| PADCEV ® | Nectin4 | IgG1 | enzyme | MMAE/ | microtubule |
| (enfortumab | | | cleavable | auristatin | inhibitor |
| vedotin-ejfv) | | | | | |
| ENHERTU ® | HER2 | IgG1 | enzyme | DXd/ | TOP1 inhibitor |
| (fam-trastuzumab | | | cleavable | camptothecin | |
| deruxtecan-nxki) | | | | | |
| TRODELVY ® | TROP2 | IgG1 | acid | SN-38/ | TOP1 inhibitor |
| (sacituzumab | | | cleavable | camptothecin | |
| govitecan-hziy) | | | | | |
| BLENREP ® | BCMA | IgG1 | non- | MMAF/ | microtubule |
| (belantamab | | | cleavable | auristatin | inhibitor |
| mafodotin-blmf) | | | | | |
| ZYNLONTA ® | CD19 | IgG1 | enzyme | SG3199/ | DNA cleavage |
| (loncastuximab | | | cleavable | PBD dimer | |
| tesirine-lpy1) | | | | | |
| TIVDAK ® | Tissue | IgG1 | enzyme | MMAE/ | microtubule |
| (tisotumab | Factor | | cleavable | auristatin | inhibitor |
| vedotin-tftv) | | | | | |

Other payloads that can be used are discussed elsewhere herein, e.g., such as the compounds discussed for use with combination therapy.

### 3. Antibody-Dependent Cell-Mediated Cytotoxicity

Antibody-dependent cellular cytotoxicity (ADCC), also called antibody-dependent cell-mediated cytotoxicity, is the process by which antibodies coat a target cell and recruit effector cells to induce target cell death via non-phagocytic mechanisms. Antibodies can bind to their specific antigens on the target cell surface via their antigen-binding fragment (Fab) portions and interact with effector cells via their fragment crystallizable region (Fc) portions thereby acting as bridges that link the effector to a target. While several classes of human antibodies can mediate ADCC, including IgG, IgA, and IgE, IgG1 is the most used subclass for cancer therapeutic antibodies. In order for an effector cell to carry out ADCC it must express Fc receptors (FcR) that will bind the antibody. The known classes of FcR include FcγR, which bind IgG; FcαR, which bind IgA; and FcεR, which bind IgE. FcγR are the most important for tumor cell clearance by myeloid cells and are comprised of activating FcγRI (CD64), FcγRIIA (CD32A), FcγRIIIA (CD16A), and inhibitory FcγRIIB (CD32B) receptors. Once the FcγR binds antibody it triggers receptor cross-linking and downstream signal propagation. Activating FcγR signal via their immunoreceptor tyrosine-based activation motifs while inhibitory FcγR signal via their immunoreceptor tyrosine-based inhibitory motifs. Many effector cells also express other receptor types such as the inhibitory killer inhibitory receptors (e.g., KIR) and activating NKG2D receptors on natural killer (NK) cells. The delicate balance amongst the activating and inhibitory pathway signaling ultimately determines effector cell response. Myeloid cells capable of acting as ADCC effectors are NK cells, monocytes, macrophages, neutrophils, eosinophils, and dendritic cells. Once these effector cells have been activated, they mediate target cell death through three key mechanisms: cytotoxic granule release, Fas signaling, and elaboration of reactive oxygen species.

Substitutions, additions, or deletions in the bispecific antibodies may be in the Fc region of the antibody and may thereby serve to modify the binding affinity of the antibody to one or more FcγR. Methods for modifying antibodies with modified binding to one or more FcγR are known in the art, see, e.g., PCT Publication Nos. WO 04/029207, WO 04/029092, WO 04/028564, WO 99/58572, WO 99/51642, WO 98/23289, WO 89/07142, WO 88/07089, and U.S. Pat. Nos. 5,843,597 and 5,642,821. In some embodiments, the invention encompasses antibodies that have altered affinity for an activating FcγR, e.g., FcγRIIIA. Preferably such modifications also have an altered Fc-mediated effector function. Modifications that affect Fc-mediated effector function are well known in the art (see U.S. Pat. No. 6,194,551, and WO 00/42072). In one particular embodiment, the modification of the Fc region results in an antibody with an altered antibody-mediated effector function, an altered binding to other Fc receptors (e.g., Fc activation receptors), an altered antibody-dependent cell-mediated cytotoxicity (ADCC) activity, an altered C1q binding activity, an altered complement-dependent cytotoxicity activity (CDC), a phagocytic activity, or any combination thereof.

### B. Inhibitors of Trogocytosis

Trogocytosis is also involved in attenuating therapeutic efficacy in tumor therapies. Thus, compositions selectively targeting this process to limit the transfer of membrane-bound proteins from a donor immune cell to a recipient cancer cell are also described and can be used alone or in combination with the disclosed antibody-based therapies and/or other cancer treatments. In preferred embodiments, compositions include one or more inhibitors of trogocytosis are suitable for reversing the effects of trogocytosis and/or enhancing therapeutic efficacy in tumor therapies in a subject. In some embodiments, the treatment presence or expression of one or more immune cell markers on the surface of the cancer cells.

In some embodiments, the compositions include one or more inhibitors that can inhibit the process of trogocytosis between immune cells and tumor cells in the tumor microenvironment and/or are combined with traditional cancer therapeutic approaches. In preferred embodiments, one or more inhibitors are used to improve therapeutic efficacy of one or more conventional tumor therapies. In some embodiments, the subject is undergoing a conventional treatment for a particular cancer, for example, surgery, transplant surgery, a radiation therapy, or chemotherapy. In other embodiments, the subject is undergoing an immunotherapy such inhibition of checkpoint proteins such as components of the PD-1/PD-L1 axis or CD28-CTLA-4 axis using one or more immune checkpoint modulators (e.g., PD-1 antagonists, PD-1 ligand antagonists, and CTLA4 antagonists), adoptive T cell therapy, and/or a cancer vaccine.

Results of Aucher et al. (Aucher A, Magdeleine E, Joly E, Hudrisier D. Blood (2008) 111(12):5621-8) showed that the trogocytosis by CD8+ and CD4+ T cells was partially or completely inhibited by inhibitors of cell activation, such as actin polymerization, kinase (such as Src-kinase, Syk-kinase, and PI3K). In addition, it was reported that amoebic trogocytosis could be inhibited by treatment with cytochalasin D, Gal/GalNAc (D-galactose/N-acetyl-D-galactosamine) lectin inhibitor, PI3K inhibitor, amoebic AGC kinase 1 (EhAGCK1) inhibitor or the mutation (Somlata, Nakada-Tsukui K, Nozaki T. Nat Commun (2017) 8(1):101), amoebic cysteine protease (EhCP) inhibitor, amoebic C2 domain protein kinase (EhC2PK) inhibitor, or mutation at low temperature (4° C.), which led to a reduction in the killing rate of host cells (Ralston K S, et al., Nature (2014) 508(7497):526-30). Among them, the AGC kinase family affects actin dynamics by manipulating the downstream PI3K, which influences the action of trogocytosis. The process of trogocytosis requires the participation of physiological temperature, actin rearrangement, Gal/GalNAc lectins, related enzymes (such as Src-kinase, Syk-kinase, and PI3K), and PI3K signals.

Pham et al. showed that the interruption of actin polymerization and lack of energy would block the process of trogocytosis, and blocking PI3K activity delayed the process of trogocytosis (Pham T, Mero P, Booth J W. PloS One (2011) 6(1):e14498). Alternatively, the inhibition of Src kinases activity slowed the process and reduced the degree of trogocytosis. Previous studies have shown that inhibitors of trogocytosis mainly include ATPase inhibitors, actin skeleton blockers, Src, Syk, PIK3 pathway kinase inhibitors, and acidification inhibitors (Gary R, et al., J Immunol (2012) 188(2):744-52, 92, 107; Gilmartin A A, Ralston K S, Petri W A Jr, mBio (2017) 8(4):e01187-17). Concanamycin A (an ATPase inhibitor) abrogated Ag-specific trogocytosis. Cytochalasin D (actin skeleton blocker) promoted actin depolymerization. Wortmannin (PI3K inhibitor) and PP2 (tyrosine-protein kinase Src inhibitor) significantly inhibited the trogocytosis by CD4+ and CD8+ T cells. Picetanannol (a tyrosine-protein kinase Syk inhibitor) inhibited the trogocytosis by T cells and neutrophils (Aljona L, Jose M. Pomona: California State Polytech Univ (2020)). Ammonium chloride (an acidification inhibitor) reduced amoebic trogocytosis and cell killing but did not weaken initiation while inhibiting the process of receptor dependence.

Thus, in some embodiments, the one or more inhibitors of trogocytosis include Gal/GalNAc (D-galactose/N-acetyl-D-galactosamine) lectin inhibitors, PI3K/AKT/mTOR pathway inhibitors, amoebic AGC kinase 1 (EhAGCK1) inhibitors, tyrosine-protein kinase Syk inhibitors, ATPase inhibitors, or combinations thereof.

In some embodiments, exemplary inhibitors of PI3K/AKT/mTOR pathway include, but are not limited to, BEZ235, LY294002, GDC-0941, BYL719, GSK2636771, TGX-221, AS25242, CAL-101, IPI-145, MK-2206, GSK690693, GDC-0068, A-674563, CCT128930, AZD8055, INK128, rapamycin, PF-04691502, everolimus, BI-D1870, H89, PF-4708671, FMK, AT7867, NU7441, PI-103, NU7026, PIK-75, ZSTK474, and PP-121.

### IV. Pharmaceutical Formulations

Any of the disclosed compounds can be formulated in a pharmaceutical composition. Pharmaceutical compositions may be formulated in a conventional manner using one or more physiologically acceptable carriers including excipients and auxiliaries which facilitate processing of the active compounds into preparations which can be used pharmaceutically. Proper formulation is dependent upon the route of administration chosen. In some embodiments, the compositions are formulated for parenteral delivery. In preferred embodiments, the compositions are formulated for local injection. Typically, the compositions will be formulated in sterile saline or buffered solution for injection into the tissues or cells to be treated. The compositions can be stored lyophilized in single use vials for rehydration immediately before use. Other means for rehydration and administration are known to those skilled in the art.

In some embodiments, the compositions are administered locally, for example, by injection directly into a site to be treated (e.g., into a tumor). In some forms, the compositions are injected or otherwise administered directly into the vasculature onto vascular tissue at the intended site of treatment. In some forms, the compositions are injected or otherwise administered directly into the vasculature onto vascular tissue adjacent to the intended site of treatment. Typically, local administration causes an increased localized concentration of the compositions which is greater than that which can be achieved by systemic administration, preferably with less toxicity and/or side effects. Targeting of the molecules or formulation can be used to achieve more selective delivery. In some embodiments, the compositions are administered directly into a tumor or into a tumor microenvironment. In some embodiments, the compositions are administered locally at the site of surgery, e.g., into the site of surgical resection at the time of surgery.

In some embodiments, pharmaceutical formulations contain bispecific or multispecific antibodies having a Fc effector region for mediating ADCC and/or conjugated to one or more cytotoxic agents, in combination with one or more pharmaceutically acceptable excipients. In other embodiments, pharmaceutical formulations contain one or more inhibitors of trogocytosis in combination with one or more pharmaceutically acceptable excipients. Representative excipients include solvents, diluents, pH modifying agents, preservatives, antioxidants, suspending agents, wetting agents, viscosity modifiers, tonicity agents, stabilizing agents, and combinations thereof. Suitable pharmaceutically acceptable excipients are preferably selected from materials which are generally recognized as safe (GRAS), and may be administered to an individual without causing undesirable biological side effects or unwanted interactions. See, for example, Remington's Pharmaceutical Sciences, 20th ed., Lippincott Williams & Wilkins, Baltimore, MD, 2000, p. 704.

The compositions are preferably formulated in dosage unit form for ease of administration and uniformity of dosage. The phrase “dosage unit form” refers to a physically discrete unit of conjugate appropriate for the patient to be treated. It will be understood, however, that the total single administration of the compositions will be decided by the attending physician within the scope of sound medical judgment. The therapeutically effective dose can be estimated initially either in cell culture assays or in animal models, usually mice, rabbits, dogs, or pigs. The animal model is also used to achieve a desirable concentration range and route of administration. Such information should then be useful to determine useful doses and routes for administration in humans. Therapeutic efficacy and toxicity of conjugates can be determined by standard pharmaceutical procedures in cell cultures or experimental animals, e.g., ED50 (the dose is therapeutically effective in 50% of the population) and LD50 (the dose is lethal to 50% of the population). The dose ratio of toxic to therapeutic effects is the therapeutic index and it can be expressed as the ratio, LD50/ED50. Pharmaceutical compositions which exhibit large therapeutic indices are preferred. The data obtained from cell culture assays and animal studies can be used in formulating a range of dosages for human use.

Pharmaceutical compositions formulated for administration by parenteral (intramuscular, intraperitoneal, intravenous, or subcutaneous injection) and enteral routes of administration are described.

### A. Parenteral Administration

The phrases “parenteral administration” and “administered parenterally” are art-recognized terms, and include modes of administration other than enteral and topical administration, such as injections, and include, without limitation, intravenous, intramuscular, intrapleural, intravascular, intrapericardial, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrastemal injection and infusion. The compositions can be administered parenterally, for example, by subdural, intravenous, intrathecal, intraventricular, intraarterial, intra-amniotic, intraperitoneal, or subcutaneous routes. In preferred embodiments, the compositions are administered via subcutaneous injection.

For liquid formulations, pharmaceutically acceptable carriers may be, for example, aqueous or non-aqueous solutions, suspensions, emulsions, or oils. Parenteral vehicles (for subcutaneous, intravenous, intraarterial, or intramuscular injection) include, for example, sodium chloride solution, Ringer's dextrose, dextrose and sodium chloride, lactated Ringer's and fixed oils. Examples of non-aqueous solvents are propylene glycol, polyethylene glycol, and injectable organic esters such as ethyl oleate. Aqueous carriers include, for example, water, alcoholic/aqueous solutions, cyclodextrins, emulsions or suspensions, including saline and buffered media. The compositions can also be administered in an emulsion, for example, water in oil. Examples of oils are those of petroleum, animal, vegetable, or synthetic origin, petrolatum, and mineral. Suitable fatty acids for use in parenteral formulations include, for example, oleic acid, stearic acid, and isostearic acid. Ethyl oleate and isopropyl myristate are examples of suitable fatty acid esters.

Formulations suitable for parenteral administration can include antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Intravenous vehicles can include fluid and nutrient replenishers, electrolyte replenishers such as those based on Ringer's dextrose. In general, water, saline, aqueous dextrose and related sugar solutions, and glycols such as propylene glycols or polyethylene glycol are preferred liquid carriers, particularly for injectable solutions.

Injectable pharmaceutical carriers for injectable compositions are well-known to those of ordinary skill in the art (see, e.g., Pharmaceutics and Pharmacy Practice, J.B. Lippincott Company, Philadelphia, PA, Banker and Chalmers, eds., pages 238-250 (1982), and ASHP Handbook on Injectable Drugs, Trissel, 15th ed., pages 622-630 (2009)).

### B. Enteral Administration

The compositions can be administered enterally. The carriers or diluents may be solid carriers such as capsule or tablets or diluents for solid formulations, liquid carriers or diluents for liquid formulations, or mixtures thereof.

For liquid formulations, pharmaceutically acceptable carriers may be, for example, aqueous or non-aqueous solutions, suspensions, emulsions, or oils. Examples of non-aqueous solvents are propylene glycol, polyethylene glycol, and injectable organic esters such as ethyl oleate. Aqueous carriers include, for example, water, alcoholic/aqueous solutions, cyclodextrins, emulsions or suspensions, including saline and buffered media.

Examples of oils are those of petroleum, animal, vegetable, or synthetic origin, for example, peanut oil, soybean oil, mineral oil, olive oil, sunflower oil, fish-liver oil, sesame oil, cottonseed oil, corn oil, olive, petrolatum, and mineral. Suitable fatty acids for use in parenteral formulations include, for example, oleic acid, stearic acid, and isostearic acid. Ethyl oleate and isopropyl myristate are examples of suitable fatty acid esters.

Vehicles include, for example, sodium chloride solution, Ringer's dextrose, dextrose and sodium chloride, lactated Ringer's and fixed oils. Formulations include, for example, aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Vehicles can include, for example, fluid and nutrient replenishers, electrolyte replenishers such as those based on Ringer's dextrose. In general, water, saline, aqueous dextrose and related sugar solutions are preferred liquid carriers. These can also be formulated with proteins, fats, saccharides and other components of infant formulas.

In further embodiments, the compositions are formulated for oral administration. Oral formulations may be in the form of chewing gum, gel strips, tablets, capsules, or lozenges. Encapsulating substances for the preparation of enteric-coated oral formulations include cellulose acetate phthalate, polyvinyl acetate phthalate, hydroxypropyl methylcellulose phthalate and methacrylic acid ester copolymers. Solid oral formulations such as capsules or tablets are preferred. Elixirs and syrups also are well known oral formulations.

### V. Methods of Use

Methods of using the disclosed compositions in a subject in need thereof are described.

Methods of using compositions including the disclosed antibodies that selectively bind to one or more distinct cell surface molecules present on the tumor cells are described. The methods provide an effective amount of one or more pharmaceutical compositions to treat and/or alleviate one or more symptoms associated with cancer, preferably reducing tumor size and/or preventing tumor growth.

Methods of using compositions for reducing or inhibiting trogocytosis in cancer cells are also described. Methods can also include administering one or more pharmacological compositions suitable for reducing or inhibiting trogocytosis in tumor cells in a subject. The methods provide an effective amount of one or more pharmacological compositions to reduce or inhibit the transfer of cell membrane and/or any molecules associated therewith from one or more immune cells that come into contact with the tumor cells in the subject, preferably, in an amount effective to increase the therapeutic efficacy of conventional cancer therapy or immune check point inhibitors.

In certain embodiments, the disclosed compositions are administered locally, for example, by injection directly into a site to be treated. In some embodiments, the compositions are injected, topically applied, or otherwise administered directly into the vasculature onto vascular tissue at or adjacent to a site of cancerous growth. For example, in embodiments, the compositions are topically applied to vascular tissue that is exposed, during a surgical procedure. Typically, local administration causes an increased localized concentration of the compositions, which is greater than that which can be achieved by systemic administration.

### A. Conditions to be Diagnosed and Treated

Methods of treating diseases and/or disorders in a subject in need thereof are provided. The subject to be treated can have a disease, disorder, or condition such as but not limited to, cancer, an immune system disorder such autoimmune disease, an inflammatory disease, an infectious disease, or combinations thereof. The disease, disorder, or condition can be associated with an elevated expression or specific expression of an antigen.

Typically, the cancer is one where the cancer has one or more immune cell markers such as, but not limited to, CD45 (CD45RA and/or CD45RO), CD14, CD16, CD56, CD68, CD4, CD53, PTPRC, FCGR2A, FCGR3A, and/or CTSS present on its surface, optionally transferred from an immune cell via trogocytosis. In some embodiments, the cancer (e.g., cancer or tumor cells thereof) was analyzed and the one or more cancer antigens were detected on the cancer cells, optionally leading to a cancer diagnosis. Additionally or alternatively, the cancer (e.g., cancer or tumor cells thereof) was analyzed and the one or more immune cell markers were detected on the cancer cells. Thus, in some embodiments, the cancer was already diagnosed and/or characterized prior to or as an adjunct to treatment.

In general, methods of administering the disclosed compositions or pharmaceutical formulations thereof are useful in the context of treating cancer, including tumor therapy. All the methods described can include the step of identifying and selecting a subject in need of treatment, or a subject who would benefit from administration with the compositions.

Typically, the subjects to be treated have a proliferative disease, such as a benign or malignant tumor. In some embodiments, the subjects to be treated have been diagnosed with stage I, stage II, stage III, or stage IV cancer.

The term cancer refers specifically to a malignant tumor. In addition to uncontrolled growth, malignant tumors exhibit metastasis. In this process, small clusters of cancerous cells dislodge from a tumor, invade the blood or lymphatic vessels, and are carried to other tissues, where they continue to proliferate. In this way a primary tumor at one site can give rise to a secondary tumor at another site.

The compositions and methods are useful for treating subjects having benign or malignant tumors by delaying or inhibiting the growth of a tumor in a subject, reducing the growth or size of the tumor, inhibiting, or reducing metastasis of the tumor, and/or inhibiting or reducing symptoms associated with tumor development or growth.

Malignant tumors that may be treated are classified according to the embryonic origin of the tissue from which the tumor is derived. Carcinomas are tumors arising from endodermal or ectodermal tissues such as skin or the epithelial lining of internal organs and glands. The compositions are particularly effective in treating carcinomas. Sarcomas, which arise less frequently, are derived from mesodermal connective tissues such as bone, fat, and cartilage. The leukemias and lymphomas are malignant tumors of hematopoietic ceils of the bone marrow. Leukemias proliferate as single cells, whereas lymphomas tend to grow as tumor masses. Malignant tumors may show up at numerous organs or tissues of the body to establish a cancer.

The types of cancer that can be treated with the provided compositions and methods include, but are not limited to, cancers such as colorectal cancer, peritoneal carcinomatosis, pancreatic cancer, multiple myeloma, sarcomas, brain, breast, esophageal, liver, lung, stomach, and uterine. In some embodiments, the compositions are used to treat multiple cancer types concurrently. The compositions can also be used to treat metastases or tumors at multiple locations. In some embodiments, the cancers to be treated are hepatocellular carcinoma, cholangiocarcinoma and medulloblastoma. In preferred embodiments, the cancers to be treated are prostate cancer, ovarian, breast, and bladder cancer.

Exemplary cancers that can be treated include brain tumors including, but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, primary brain lymphoma; breast cancer including, but not limited to, adenocarcinoma, lobular (small cell) carcinoma, intraductal carcinoma, medullary breast cancer, mucinous breast cancer, tubular breast cancer, papillary breast cancer, Paget's disease, and inflammatory breast cancer; adrenal cancer, including, but not limited to, pheochromocytoma and adrenocortical carcinoma; thyroid cancer such as but not limited to papillary or follicular thyroid cancer, medullary thyroid cancer and anaplastic thyroid cancer; pancreatic cancer, including, but not limited to, insulinoma, gastrinoma, glucagonoma, vipoma, somatostatin-secreting tumor, and carcinoid or islet cell tumor; pituitary cancers including, but not limited to, Cushing's disease, prolactin-secreting tumor, acromegaly, and diabetes insipidus; eye cancers including, but not limited to, ocular melanoma such as iris melanoma, choroidal melanoma, and ciliary body melanoma, and retinoblastoma; vaginal cancers, including, but not limited to, squamous cell carcinoma, adenocarcinoma, and melanoma; vulvar cancer, including, but not limited to, squamous cell carcinoma, melanoma, adenocarcinoma, basal cell carcinoma, sarcoma, and Paget's disease; cervical cancers including, but not limited to, squamous cell carcinoma, and adenocarcinoma; uterine cancers including, but not limited to, endometrial carcinoma and uterine sarcoma; ovarian cancers including, but not limited to, ovarian epithelial carcinoma, borderline tumor, germ cell tumor, and stromal tumor; esophageal cancers including, but not limited to, squamous cancer, adenocarcinoma, adenoid cystic carcinoma, mucoepidermoid carcinoma, adenosquamous carcinoma, sarcoma, melanoma, plasmacytoma, verrucous carcinoma, and oat cell (small cell) carcinoma; stomach cancers including, but not limited to, adenocarcinoma, fungating (polypoid), ulcerating, superficial spreading, diffusely spreading, malignant lymphoma, liposarcoma, fibrosarcoma, and carcinosarcoma; colon cancers; rectal cancers; liver cancers including, but not limited to, hepatocellular carcinoma and hepatoblastoma, gallbladder cancers including, but not limited to, adenocarcinoma; cholangiocarcinomas including, but not limited to, papillary, nodular, and diffuse; lung cancers including, but not limited to, non-small cell lung cancer, squamous cell carcinoma (epidermoid carcinoma), adenocarcinoma, large-cell carcinoma and small-cell lung cancer; testicular cancers including, but not limited to, germinal tumor, seminoma, anaplastic, classic (typical), spermatocytic, nonseminoma, embryonal carcinoma, teratoma carcinoma, choriocarcinoma (yolk-sac tumor); prostate cancers including, but not limited to, adenocarcinoma, leiomyosarcoma, and rhabdomyosarcoma; penal cancers; oral cancers including, but not limited to, squamous cell carcinoma; basal cancers; salivary gland cancers including, but not limited to, adenocarcinoma, mucoepidermoid carcinoma, and adenoid cystic carcinoma; pharynx cancers including, but not limited to, squamous cell cancer, and verrucous; skin cancers including, but not limited to, basal cell carcinoma, squamous cell carcinoma and melanoma, superficial spreading melanoma, nodular melanoma, lentigo malignant melanoma, acral lentiginous melanoma; kidney cancers including, but not limited to, renal cell cancer, adenocarcinoma, hypernephroma, fibrosarcoma, transitional cell cancer (renal pelvis and/or ureter); Wilms' tumor; bladder cancers including, but not limited to, transitional cell carcinoma, squamous cell cancer, adenocarcinoma, carcinosarcoma.

In a particular embodiment the cancer to be treated is a kidney cancer such as renal cell carcinoma (RCC). Renal cell carcinoma (RCC), also known as renal cell cancer or renal cell adenocarcinoma, is the most common type of kidney cancer. About 9 out of 10 kidney cancers are renal cell carcinomas. The RCC can be clear cell RCC or a non-clear cell RCC such as papillary renal cell carcinoma, chromophobe renal cell carcinoma, collecting duct RCC, multilocular cystic RCC, medullary carcinoma, mucinous tubular and spindle cell carcinoma, or neuroblastoma-associated RCC.

In another embodiments, the cancer is colorectal adenocarcinoma, colorectal or pancreatic neuroendocrine cancer.

In another embodiment, the cancer is triple negative breast cancer.

The methods and compositions as described are useful for both prophylactic and therapeutic treatment.

Therapeutic treatment involves administering to a subject a therapeutically effective amount of the compositions or pharmaceutically acceptable salts thereof as described after cancer is diagnosed.

In further embodiments, the compositions are used for prophylactic use i.e., prevention, delay in onset, diminution, eradication, or delay in exacerbation of signs or symptoms after onset, and prevention of relapse. For prophylactic use, a therapeutically effective amount of the compounds and compositions or pharmaceutically acceptable salts thereof as described are administered to a subject prior to onset (e.g., before obvious signs of cancer), during early onset (e.g., upon initial signs and symptoms of cancer), or after an established development of cancer. Prophylactic administration can occur for several days to years prior to the manifestation of symptoms. Prophylactic administration can be used, for example, in the chemo-preventative treatment of subjects presenting precancerous lesions, those diagnosed with early-stage malignancies, and for subgroups with susceptibilities (e.g., family, racial, and/or occupational) to particular cancers.

### B. Combination Therapies

In some embodiments, patients are also subject to one or more adjunct therapies or procedures, or can be an adjunct therapy to one or more primary therapies or producers. The additional therapy or procedure can be simultaneous or sequential with the combination therapy. In some embodiments, the additional therapy is performed between drug cycles or during a drug holiday that is part of the combination therapy dosage regime. In preferred embodiments, the additional therapy is a conventional treatment for cancer, more preferably a conventional treatment for the particular cancer type, e.g., prostate or breast cancer. For example, in some embodiments, the additional therapy or procedure is surgery, a radiation therapy, or chemotherapy. For example, in a particular embodiment, combination therapies are used simultaneously or sequentially with a regime of a chemotherapeutic agent, e.g., docetaxel or cabazitaxel. In some embodiments, the adjunct or additional therapy is part of the combination therapy.

In some embodiments, the conventional cancer therapy is in the form of one or more additional active agents. Therefore, in some embodiments, the methods administer compositions in combination with one or more additional active agents. The combination therapies can include administration of the compositions for reducing/inhibiting the activities of trogocytosis in tumor cells, and one or more additional active agents together in the same admixture, or in separate admixtures. Therefore, in some embodiments, the methods administer a pharmaceutical formulation including compositions for reducing/inhibiting the activities of trogocytosis in tumor cells as well as one, two, three, or more additional active agents. Such formulations typically include an effective amount of compositions for reducing/inhibiting the activities of trogocytosis in tumor cells, and an effective amount of an additional therapeutic, prophylactic or diagnostic agent.

In further embodiments, the combination therapies can include administration of the compositions including the disclosed bispecific antibodies with a toxic payload and/or an Fc effector function, and one or more additional active agents together in the same admixture, or in separate admixtures. Therefore, in some embodiments, the methods administer a pharmaceutical formulation including the disclosed bispecific antibodies with a toxic payload and/or an Fc effector function as well as one, two, three, or more additional active agents. Such formulations typically include an effective amount of compositions including the disclosed bispecific antibodies with a toxic payload and/or an Fc effector function, and an effective amount of an additional therapeutic, prophylactic or diagnostic agent.

The additional active agent(s) can have the same, or different mechanisms of action. In some embodiments, the combination results in an additive effect on the treatment of the cancer. In some embodiments, the combinations result in a more than additive effect on the treatment of the disease or disorder.

The additional therapy or procedure can be simultaneous or sequential with the administration of the compositions for reducing/inhibiting the activities of trogocytosis. In some embodiments the additional therapy is performed between drug cycles or during a drug holiday that is part of the composition dosage regime. For example, in some embodiments, the additional therapy or procedure is surgery, a radiation therapy, or chemotherapy.

Additional therapeutic agents include conventional cancer therapeutics such as chemotherapeutic agents, cytokines, chemokines, and radiation therapy, as discussed above. The majority of chemotherapeutic drugs can be divided into alkylating agents, antimetabolites, anthracyclines, plant alkaloids, topoisomerase inhibitors, and other antitumor agents. These drugs affect cell division or DNA synthesis and function in some way. Additional therapeutics include monoclonal antibodies and the tyrosine kinase inhibitors e.g., imatinib mesylate (GLEEVEC® or GLIVEC®), which directly targets a molecular abnormality in certain types of cancer (chronic myelogenous leukemia, gastrointestinal stromal tumors).

In some embodiments, the additional therapy is a chemotherapeutic agent. Representative chemotherapeutic agents include, but are not limited to, amsacrine, bleomycin, busulfan, camptothecin, capecitabine, carboplatin, carmustine, chlorambucil, cisplatin, cladribine, clofarabine, crisantaspase, cyclophosphamide, cytarabine, dacarbazine, dactinomycin, daunorubicin, docetaxel, doxorubicin, epipodophyllotoxins, epirubicin, etoposide, etoposide phosphate, fludarabine, fluorouracil, gemcitabine, hydroxycarb amide, idarubicin, ifosfamide, innotecan, leucovorin, liposomal doxorubicin, liposomal daunorubici, lomustine, mechlorethamine, melphalan, mercaptopurine, mesna, methotrexate, mitomycin, mitoxantrone, oxaliplatin, paclitaxel, pemetrexed, pentostatin, procarbazine, raltitrexed, satraplatin, streptozocin, teniposide, tegafur-uracil, temozolomide, teniposide, thiotepa, tioguanine, topotecan, treosulfan, vinblastine, vincristine, vindesine, vinorelbine, vorinostat, taxol, trichostatin A and derivatives thereof, trastuzumab (HERCEPTIN®), cetuximab, and rituximab (RITUXAN® or MABTHERA®), bevacizumab (AVASTIN®), and combinations thereof. Representative pro-apoptotic agents include, but are not limited to, fludarabinetaurosporine, cycloheximide, actinomycin D, lactosylceramide, 15d-PGJ(2)5, and combinations thereof.

In some embodiments, the compositions and methods are used prior to or in conjunction with an immunotherapy such as inhibition of checkpoint proteins such as components of the PD-1/PD-L1 axis or CD28-CTLA-4 axis using one or more immune checkpoint modulators (e.g., PD-1 antagonists, PD-1 ligand antagonists, and CTLA4 antagonists), adoptive T cell therapy, and/or a cancer vaccine. Exemplary immune checkpoint modulators used in immunotherapy include Pembrolizumab (anti-PD1 mAb), Durvalumab (anti-PDL1 mAb), PDR001 (anti-PD1 mAb), Atezolizumab (anti-PDL1 mAb), Nivolumab (anti-PD1 mAb), Tremelimumab (anti-CTLA4 mAb), Avelumab (anti-PDL1 mAb), and RG7876 (CD40 agonist mAb).

In some embodiments, the additional therapy is adoptive T cell therapy. Methods of adoptive T cell therapy are known in the art and used in clinical practice. Generally adoptive T cell therapy involves the isolation and ex vivo expansion of tumor-specific T cells to achieve greater number of anti-tumor T cells than what could be obtained by vaccination alone. The tumor-specific T cells are then infused into patients with cancer in an attempt to give their immune system the ability to overwhelm remaining tumor via T cells, which can attack and kill the cancer. Several forms of adoptive T cell therapy can be used for cancer treatment including, but not limited to, culturing tumor infiltrating lymphocytes or TIL; isolating and expanding one particular T cell or clone; and using T cells that have been engineered to recognize and attack tumors. In some embodiments, the T cells are taken directly from the patient's blood. Methods of priming and activating T cells in vitro for adaptive T cell cancer therapy are known in the art. See, for example, Wang, et al, Blood, 109(11):4865-4872 (2007) and Hervas-Stubbs, et al, J. Immunol., 189(7):3299-310 (2012).

Historically, adoptive T cell therapy strategies have largely focused on the infusion of tumor antigen specific cytotoxic T lymphocytes (CTL) that can directly kill tumor cells. However, CD4+ T helper (Th) cells such as Th1, Th2, Tfh, Treg, and Th17 can also be used. Th cells can activate antigen-specific effector cells and recruit cells of the innate immune system such as macrophages and dendritic cells to assist in antigen presentation by antigen presentation cells (APC), and antigen-primed Th cells can directly activate tumor antigen-specific CTL. As a result of activating APCs, antigen-specific Th1 have been implicated as the initiators of epitope or determinant spreading which is a broadening of immunity to other antigens in the tumor. The ability to elicit epitope spreading, broadens the immune response to many potential antigens in the tumor and can lead to more efficient tumor cell kill due to the ability to mount a heterogenetic response. In this way, adoptive T cell therapy can used to stimulate endogenous immunity. In some embodiments, the T cells express a chimeric antigen receptor (CARs, CAR T cells, or CARTs). Artificial T cell receptors are engineered receptors, which graft a particular specificity onto an immune effector cell. Typically, these receptors are used to graft the specificity of a monoclonal antibody onto a T cell and can be engineered to target virtually any tumor-associated antigen. First generation CARs typically had the intracellular domain from the CD3ζ-chain, which is the primary transmitter of signals from endogenous TCRs. Second generation CARs add intracellular signaling domains from various costimulatory protein receptors (e.g., CD28, 41BB, ICOS) to the cytoplasmic tail of the CAR to provide additional signals to the T cell, and third generation CARs combine multiple signaling domains, such as CD3ζ-CD28-41BB or CD3ζ-CD28-OX40, to further enhance effectiveness.

In some embodiments, the compositions and methods are used prior to or in conjunction with a cancer vaccine, for example, a dendritic cell cancer vaccine. Vaccination typically includes administering a subject an antigen (e.g., a cancer antigen) together with an adjuvant to elicit therapeutic T cells in vivo. In some embodiments, the cancer vaccine is a dendritic cell cancer vaccine in which the antigen is delivered by dendritic cells primed ex vivo to present the cancer antigen. Examples include PROVENGE® (sipuleucel-T), which is a dendritic cell-based vaccine for the treatment of prostate cancer (Ledford, et al., Nature, 519, 17-18 (5 Mar. 2015). Such vaccines and other compositions and methods for immunotherapy are reviewed in Palucka, et al., Nature Reviews Cancer, 12, 265-277 (April 2012).

In some embodiments, the compositions and methods are used prior to or in conjunction with surgical removal of tumors, for example, in preventing primary tumor metastasis. In some embodiments, the compositions and methods are used to enhance the body's own anti-tumor immune functions.

### C. Controls

The effect of the compositions can be compared to a control. Suitable controls are known in the art and include, for example, an untreated subject, or a placebo-treated subject. A typical control is a comparison of a condition or symptom of a subject prior to and after administration of the targeted agent. The condition or symptom can be a biochemical, molecular, physiological, or pathological readout. For example, the effect of the bispecific antibody composition on a particular cancer can be compared to an untreated subject, or the condition of the subject prior to treatment. In some embodiments, the symptom, pharmacologic, or physiologic indicator is measured in a subject prior to treatment, and again one or more times after treatment is initiated. In some embodiments, the control is a reference level, or average determined based on measuring the symptom, pharmacologic, or physiologic indicator in one or more subjects that do not have the disease or condition to be treated (e.g., healthy subjects). In some embodiments, the effect of the treatment is compared to a conventional treatment that is known in the art. In some embodiments, an untreated control subject suffers from the same disease or condition as the treated subject.

### D. Dosages and Effective Amounts

Dosage and dosing regimens are dependent on the severity and location of the disorder or condition and/or methods of administration, and can be determined by those skilled in the art.

Preferably, the active agents do not target or otherwise modulate the activity or quantity of healthy cells not within or associated with the diseased/damaged tissue, or do so at a reduced level compared to cells associated with the inflammation or of the tumor region. In this way, by-products and other side effects associated with the compositions are reduced.

A pharmaceutical composition including a therapeutically effective amount of the disclosed compositions and a pharmaceutically acceptable diluent, carrier or excipient is described. In some embodiments, the pharmaceutical compositions include an effective amount of monospecific or bispecific antibodies conjugated to one or more cytotoxic agents and/or effector Fc region to facilitate ADCC or CDC. In additional or alternative embodiments, the pharmaceutical compositions include an effective amount of one or more inhibitors of trogocytosis.

Dosage forms of the pharmaceutical composition including the compositions are also provided. “Dosage form” refers to the physical form of a dose of a therapeutic compound, such as a capsule or vial, intended to be administered to a patient. The term “dosage unit” refers to the amount of the therapeutic compounds to be administered to a patient in a single dose.

The actual effective amounts of disclosed compositions can vary according to factors including the specific active agent administered, the particular composition formulated, the mode of administration, and the age, weight, condition of the subject being treated, as well as the route of administration and the disease or disorder. The subjects are preferably humans. Generally, the dosage may be lower for intravenous injection or infusion.

In general, the timing and frequency of administration will be adjusted to balance the efficacy of a given treatment or diagnostic schedule with the side effects of the given delivery system. Exemplary dosing frequencies include continuous infusion, single and multiple administrations such as hourly, daily, weekly, monthly, or yearly dosing.

It will be understood by those of ordinary skill that a dosing regimen can be any length of time sufficient to treat the disorder in the subject. In some embodiments, the regimen includes one or more cycles of a round of therapy followed by a drug holiday (e.g., no drug). The drug holiday can be 1, 2, 3, 4, 5, 6, or 7 days; or 1, 2, 3, 4 weeks, or 1, 2, 3, 4, 5, or 6 months.

### VI. Kits

The compositions can be packaged in a kit. In some embodiments, the kit can include a single dose or a plurality of doses of a composition including bispecific antibodies suitable for the above disclosed antibody-target therapy in a pharmaceutically acceptable carrier for shipping and storage and/or administration, and instructions for administering the compositions. In other embodiments, the kit can include a single dose or a plurality of doses of a composition including one or more inhibitors of trogocytosis in a pharmaceutically acceptable carrier for shipping and storage and/or administration, and instructions for administering the compositions. Specifically, the instructions direct that an effective amount of the composition be administered to an individual with a particular condition/disease as indicated. The composition can be formulated as described above with reference to a particular treatment method and can be packaged in any convenient manner.

The present invention can be further understood by the following numbered paragraphs:
1. An antibody or antigen binding fragment including
   i) a first antigen-binding site, and
   ii) a second antigen-binding site,
wherein the first antigen-binding site specifically binds to an immune cell antigen present on a cancer cell, and the second antigen-binding site specifically binds to a non-immune cell cancer antigen, optionally wherein presence or expression of the immune cell antigen on the cancer cell is due to trogocytosis.
2. The antibody or antigen binding fragment of paragraph 1, wherein the cancer antigen is a cell surface molecule selected from the group consisting of 4-1BB, 5T4, adenocarcinoma antigen, alpha-fetoprotein, BAFF, BCMA, B-lymphoma cell, C242 antigen, Cancer Antigen 125 (CA-125), carbonic anhydrase 9 (CA-IX), C-MET, CCR4, CD152, CD19, CD20, CD200, CD22, CD221, CD23 (IgE receptor), CD28, CD30 (TNFRSF8), CD33, CD4, CD40, CD44 v6, CD51, CD52, CD56, CD74, CD79b, CD80, CEA, CNT0888, CTLA-4, DR5, EGFR, EpCAM, CD3, FAP, fibronectin extra domain-B, folate receptor 1, GD2, GD3 ganglioside, glycoprotein 75, glycoprotein non-metastatic B (GPNMB), HER2/neu, HGF, human scatter factor receptor kinase, IGF-1 receptor, IGF-I, IgGl, Ll-CAM, IL-13, IL-6, insulin-like growth factor I receptor, integrin α5β1, integrin ανβ3, MORAb-009, MS4A1, MUC1, mucin CanAg, N-glycolylneuraminic acid, Nectin4, NPC-1C, PDGF-R a, PDL192, phosphatidylserine, prostatic carcinoma cells, RANKL, RON, ROR1, SCH 900105, SDC1, SLAMF7, TAG-72, tenascin C, TGF beta 2, TGF-β, TRAIL-R1, TRAIL-R2, trophoblast cell surface antigen (TROP-2), tumor antigen CTAA16.88, VEGF-A, VEGFR-1, VEGFR2, and vimentin.
3. An antibody or antigen binding fragment including
   i) a first antigen-binding site, and
   ii) a second antigen-binding site,
wherein the first antigen-binding site specifically binds to an immune cell antigen present on a cancer cell, and the second antigen-binding site specifically binds to an antigen on an immune cell, optionally wherein presence or expression of the immune cell antigen on the cancer cell is due to trogocytosis, optionally wherein the cancer cell does not have the antigen on the immune cell.
4. The antibody or antigen binding fragment of paragraph 3, wherein the antigen on the immune cell is selected from B220/CD45R, CCR10, CCR127, CD2, CD3, CD4, CD5, CD8, CD11a, CD19, CD20, CD25, CD27, CD28, CD31, CD34, CD38, CD45RA, CD45RO, CD56, CD58, CD62L, CD103, CD122, CD161, CD194, CD195, CD196, CD197, CD294, Fc gamma RIII (CD16), Fc gamma RIIIA/CD16a, Fc gamma RIIIB/CD16b, Flt-3/Flk-2, IL-7R alpha/CD127, NCAM-1/CD56, Neprilysin/CD10, NKp46/NCR1, Siglec-2/CD22, and those listed in Table 1.
5. The antibody or antigen binding fragment of paragraphs 3 or 4, wherein the antibody is a Bispecific T cell engager (BiTE).
6. The antibody or antigen binding fragment of any one of paragraphs 1-5, wherein immune cell antigen present on the cancer cell is a cell surface molecule selected from the group consisting of CD45, CD56, CD16, CD14, CD68, CD4, CD53, PTPRC, FCGR2A, FCGR3A, and CTSS.
7. The antibody or antigen binding fragment of any one of paragraphs 1-6, wherein the antibody or antigen binding fragment is a bispecific, trispecific, or multispecific antibody.
8. The antibody or antigen binding fragment of any one of paragraphs 1-7, wherein the antibody or antigen binding fragment is a bispecific antibody.
9. The antibody or antigen binding fragment of any one of paragraphs 1-8, wherein the antibody or antigen binding fragment is conjugated to one of more of cytotoxic agents, drugs, receptors, enzymes, receptor ligands.
10. The antibody or antigen binding fragment of any one of paragraphs 1-9, wherein the antibody or antigen binding fragment is conjugated to a cytotoxic agent.
11. The antibody or antigen binding fragment of paragraph 10, wherein the cytotoxic agent is selected from the group consisting of anti-microtubules agents and DNA-damaging agents.
12. The antibody or antigen binding fragment of paragraph 10 or 11, wherein the cytotoxic agent is selected from the from the group consisting of maytansinoids, auristatins, and calicheamicins.
13. The antibody or antigen binding fragment of any one of paragraphs 1-8, wherein the antibody or antigen binding fragment includes an Fc fragment that facilitates binding to a Fc receptor on an immune effector cell.
14. The antibody or antigen binding fragment of paragraph 13, wherein the Fc receptor is FcγR.
15. The antibody or antigen binding fragment of paragraph 13, wherein the immune effector cell is selected from NK cells, monocytes, macrophages, neutrophils, eosinophils, and dendritic cells.
16. The antibody or antigen binding fragment of any one of paragraphs 13-15, wherein the binding to Fc receptor facilitates cytotoxic granule release, Fas signaling, elaboration of reactive oxygen species, or combinations thereof.
17. A pharmaceutical composition including a therapeutically effective amount of the antibody or antigen binding fragment of any one of paragraphs 1-16.
18. The pharmaceutical composition of paragraph 17, formulated for intravenous, intrathecal, intratumoral, or oral administration.
19. The pharmaceutical composition of paragraph 17 or 18, formulated for local or topical administration.
20. A method of treating cancer including administering a subject with cancer with an effective amount of a pharmaceutical composition including an antibody or antigen binding fragment that specially binds to an immune cell antigen present on a cancer cell, optionally wherein presence or expression of the immune cell antigen on the cancer cell is due trogocytosis.
21. The method of paragraph 20, wherein immune cell antigen present on the cancer cell is a cell surface molecule selected from the group consisting of CD45, CD56, CD16, CD14, CD68, CD4, CD53, PTPRC, FCGR2A, FCGR3A, CTSS, and combinations thereof.
22. The method of paragraph 21, wherein the antibody or fragment thereof is a monospecific, bispecific, or multispecific.
23. The method of paragraphs 21 or 22, wherein the antibody or fragment is a BiTE.
24. The method of paragraphs 21 or 22, wherein the antibody or fragment thereof is conjugated to one of more of cytotoxic agents, drugs, receptors, enzymes, receptor ligands.
25. The method of paragraph 24, wherein the antibody or fragment thereof is conjugated to a cytotoxic agent.
26. The method of paragraph 25, wherein the cytotoxic agent is selected from the group consisting of anti-microtubules agents and DNA-damaging agents.
27. The method of paragraph 25 or 26, wherein the cytotoxic agent is selected from the from the group consisting of maytansinoids, auristatins, and calicheamicins.
28. The method of any one of paragraphs 20-27, wherein the antibody or fragment thereof is conjugated to a Fc fragment that facilitates binding to a Fc receptor on an immune effector cell.
29. The method of paragraph 28, wherein the Fc receptor is FcγR.
30. The method of paragraphs 28 or 29, wherein the immune effector cell is selected from NK cells, monocytes, macrophages, neutrophils, eosinophils, and dendritic cells.
31. The method of any one of paragraphs 28-30, wherein the binding to Fc receptor facilitates cytotoxic granule release, Fas signaling, elaboration of reactive oxygen species, or combinations thereof.
32. A method of treating cancer in a subject in need thereof including
   administering to the subject an effective amount of the pharmaceutical composition of any one of paragraphs 17-19 to treat or alleviate one or more symptoms of the cancer in the subject.
33. The method of any one of paragraphs 20-32, wherein the cancer is breast cancer, head and neck squamous cell carcinoma, glioblastoma, pancreatic cancer, lung cancer, melanoma, ovarian cancer, and sarcoma.
34. The method of any one of paragraphs 20-33, wherein the cancer was resistant or insensitive to one or more conventional chemotherapeutic agents and/or immunotherapy, prior to the administration of the pharmaceutical composition.
35. The method of any one of paragraphs 32-34, further including administering one or more conventional chemotherapeutic agents and/or immunotherapy.
36. The method of paragraph 35, wherein the immunotherapy is one or more check point inhibitors selected from the group consisting of PD-1 antagonists, PD-1 ligand antagonists, and CTLA4 antagonists.
37. The method of paragraph 35, wherein the one or more conventional chemotherapeutic agents are selected from the group consisting of alkylating agents, antimetabolites, anthracyclines, plant alkaloids, topoisomerase inhibitors, and other anti-tumor agents.
38. The method of any one of paragraphs 35-37, wherein the administration of the one or more conventional chemotherapeutic agents and/or immunotherapy is simultaneous or sequential with the administration of the pharmaceutical composition.
39. The method of any one of paragraphs 20-38, wherein the effective amount of the pharmaceutical composition is effective to reduce or inhibit proliferation, migration, invasion, motility, and/or metastatic abilities of the cancer cells.
40. The method of any one of paragraphs 20-39, wherein the pharmaceutical composition is administered to the subject locally to the site of surgery resection at the time of surgery.
41. A method of characterizing non-immune cells including detecting one or more immune cell markers on non-immune cells of a sample from a subject.
42. The method of paragraph 41, wherein the non-immune cells are suspected or known to be cancer cells.
43. The method of paragraph 42, wherein the non-immune cells are from a biopsy.
44. The method of any one of paragraphs 41-43 further including detecting one or more cancer antigens that are not immune cell marker(s).
45. The method of any one of paragraphs 41-44, wherein detection includes an immunoassay including an antibody or antibodies specific to the immune cell marker and/or cancer antigen.
46. The method of any one of paragraphs 41-44, wherein detection of the immune cell marker indicates the cancer cells were subject to trogocytosis.
47. The method of any one of paragraphs 41-46 further including treating the subject for cancer.
48. The method of paragraph 47, wherein the treatment includes treating the subject with the pharmaceutical composition of any one of paragraphs 17-19.
49. The method of paragraphs 47 or 48, where the treatment includes treating the subject with an inhibitor of trogocytosis.
50. The method of paragraph 49, wherein the inhibitor of trogocytosis includes Gal/GalNAc (D-galactose/N-acetyl-D-galactosamine) lectin inhibitors, PI3K/AKT/mTOR pathway inhibitors, amoebic AGC kinase 1 (EhAGCK1) inhibitors, tyrosine-protein kinase Syk inhibitors, ATPase inhibitors, or combinations thereof.
51. The method of any one of paragraphs 47-50 including treating the subject with surgery, a radiation therapy, chemotherapy, and/or immunotherapy optionally inhibition of one or more checkpoint proteins and/or adoptive cell therapy.
52. The method of any one of paragraphs 47-51 further including detecting the immune cell marker(s) in one or more subsequent sample(s) taken after treatment, wherein a reduction of the immune cell marker(s) over time is an indication that the treatment is effective.
53. The method of any one of paragraphs 47-52 wherein the immune cell marker(s) is selected from CD45 optionally CD45RA and/or CD45RO, CD14, CD16, CD56, CD68, CD4, CD53, PTPRC, FCGR2A, FCGR3A, and/or CTSS.
54. The method of any one of paragraphs 42-53, wherein the cancer antigen is carbonic anhydrase 9 (CAIX), and optionally wherein the cancer is kidney cancer optionally a renal cell carcinoma.
55. A method of identifying a compound that reduces trogocytosis and/or treats cancer including detecting one or more immune cell markers on cancer cells prior and after treatment with a test compound, and selecting the compound as effective for reducing trogocytosis and/or treating cancer if the immune cell markers is reduced after treatment relative to before treatment.
56. The method of paragraph 55 wherein the treatment is in vivo, in vitro, or ex vivo.

The present invention will be further understood by reference to the following non-limiting examples.

### EXAMPLES

### Example 1: RCC Tumors Show Widespread Expression of Trogocytic Markers

### Material and Methods

### Immunofluorescent Staining Assays

The following primary antibodies were used in immunofluorescent experiments: anti-human CAIX (R&D Biosystems, AF2188), CD45RA (Sino Biological Inc., 102580-T08), CD56 (PROTEINTECH®, 14255-1-AP), CD68 (Sino Biological Inc., 11192-T26), CD16 (PROTEINTECH®, 16559-1-AP), CD14 (R&D Biosystems, BAF383). Paraffin-embedded tissue slides were obtained from Tissue Acquisition and Cellular/Molecular Analysis (TACMASR) core facility at the University of Arizona. Slides were deparaffinized for 9 min in Xylenes. They were then washed in decreasing concentrations of ethanol solutions at 1 min intervals for 5 min. The slides were then washed in MilliQH2O for 10 minutes. Then, antigen retrieval was performed using boiling Antigen Unmasking Solution (Vector Laboratories, H-3300) for 10 min and left to cool for an additional 20 min. Slides were blocked with 20% donkey serum/1×DPBS-0.1% Tween-20 for 30 min. Primary antibody solutions were prepared according to their recommended dilutions. Slides were incubated with primary antibodies overnight at 4° C. and washed with 1×DPBS-0.1% Tween-20 for 5 min. Samples were quenched using Invitrogen READYPROBES™ Tissue Autofluorescence Quenching Kit for 3-5 min. Secondary anti-donkey antibodies from Invitrogen were applied and incubated for 1 hour at room temperature in the dark. Samples were quenched an additional time and stained with Hoescht nuclear stain for 10 min. Coverslips were mounted on the stained slides using FLUOROMOUNT-G™ (Invitrogen, 00-4958-02). Slides were imaged using the Echo Inc. Revolution LED microscope.

### Immunofluorescent Image Quantification

Images used for analysis were taken at 40× on an Echo Inc. Revolution LED microscope. Images were exported in separate channels into ImageJ (FIJI) for quantification. Nuclei were identified using the StarDist2D plugin (Schmidt, U., et al., Cell Detection with Star-Convex Polygons, in Medical Image Computing and Computer Assisted Intervention—MICCAI 2018. 2018. p. 265-273), then a Voronoi algorithm was applied to estimate the boundaries of individual cells. The tumor marker channel was used to subtract all non-tumor cells from the Voronoi cell map. The resulting regions of interest (ROIs) were then saved to be used as a map for all tumor cells in each image. The channel containing the trogocytic marker of interest was then run through a background subtraction algorithm and the resulting signal was normalized using the CLAHE histogram normalization plugin. The channel was then converted to a binary image and the ROIs previously described were applied. The total surface area of each cell was measured to give a two-dimensional representation of the cellular surface area being occupied by a trogocytic marker. Cells with trogocytic markers occupying over 55% and less than 90% of their total 2D surface area were considered trogocytic. These cutoffs were determined to exclude autofluorescence, other sources of background signal, and infiltrating immune cells from quantification. It is believe this method of quantification is quite conservative and may exclude low expressing trogocytic tumor cells, implying that the true percentage of tumor cells that are trogocytic may be much higher than this analysis estimates. This could account for the difference between the IF quantification and flow cytometry results.

Associated Codes

```
  Code 1
{run(“Enhance Contrast...”, “saturated=0.35 normalize”);
run(“CLAHE ”, “blocksize=127 histogram=256 maximum=3”);
run(“Trainable Weka Segmentation”);
selectWindow(“Trainable Weka Segmentation v3.3.2”);
call(“trainableSegmentation.Weka_Segmentation.loadClassifier”,
“C:\\Users\\Owner\\Desktop\\classifier.model”);
call(“trainableSegmentation.Weka_Segmentation.getProbability”);
run(“Duplicate...”, “use”);
setOption(“BlackBackground”, false);
run(“Convert to Mask”);
run(“Invert”);
run(“Analyze Particles...”, “size=20-500 display clear summarize overlay
add”);run(“Enhance Contrast...”, “saturated=0.35 normalize”);
run(“CLAHE ”, “blocksize=127 histogram=256 maximum=3”);
run(“Trainable Weka Segmentation”);
selectWindow(“Trainable Weka Segmentation v3.3.2”);
call(“trainableSegmentation.Weka_Segmentation.loadClassifier”,
“C:\\Users\\Owner\\Desktop\\classifier.model”);
call(“trainableSegmentation.Weka_Segmentation.getProbability”);
run(“Duplicate...”, “use”);
setOption(“BlackBackground”, false);
run(“Convert to Mask”);
run(“Invert”);
run(“Analyze Particles...”, “size=20-500 display clear summarize overlay
add”);}
  Code 2
{run(“CLAHE ”, “blocksize=127 histogram=256 maximum=3”);
setOption(“BlackBackground”, false);
run(“Convert to Mask”);}
  Code 3
macro “Nuclear Voronoi [o]”{rename(“nuc”);
run(“Command From Macro”,
“command=[de.csbdresden.stardist.StarDist2D], args=[‘input’:‘nuc’,
‘modelChoice’:‘Versatile (fluorescent nuclei)’, ‘normalizeInput’:‘true’,
‘percentileBottom’:‘1.0’, ‘percentileTop’:‘99.8’, ‘probThresh’:‘0.45’,
‘nmsThresh’:‘0.6000000000000001’, ‘outputType’:‘Both’, ‘nTiles’:‘1’,
‘excludeBoundary’:‘2’, ‘roiPosition’:‘Automatic’, ‘verbose’:‘false’,
‘showCsbdeepProgress’:‘false’, ‘showProbAndDist’:‘false’],
process=[false]”);
selectWindow(“Label Image”);
close( );
roiManager(“Deselect”);
roiManager(“Combine”);
setBackgroundColor(0, 0, 0);
run(“Clear Outside”);
setAutoThreshold(“Default dark no-reset”);
//run(“Threshold...”);
setThreshold(2, 65535, “raw”);
run(“Close”);
setOption(“BlackBackground”, true);
run(“Convert to Mask”);
run(“Watershed”);
//select nuclei_mask
run(“Analyze Particles...”, “add”);
// here we make the Voronoi
run(“Voronoi”);
rename(“voronoi”);
selectImage(“voronoi”);
setThreshold(1, 255);
run(“Convert to Mask”);
```

### Flow Cytometry Assays

The following conjugated antibodies were used in flow cytometric experiments: CD45 (clone 2D1, BioLegend, 368532), CD16 (clone CB16, Invitrogen, 17-0168-42), CD14 (clone 61D3, Invitrogen, 414-0149-42), CD45RO (clone UCHL1, Invitrogen, 12-0457-42), CD45RA (clone HI100, Invitrogen, 17-0458-42), CD44 (clone IM7, Invitrogen, 404-0441-82), CAIX (R&D Biosystems, FAB2188G), CD56 (clone CMSSB, Invitrogen, 12-0567-42), and CD68 (clone REA886, Miltenyi Biotec, 130-114-463). Fresh tumor tissue was acquired through the Tissue Acquisition and Cellular/Molecular Analysis (TACMASR) core facility at the University of Arizona. Tumor samples were finely minced into 1 mm² sections and washed with DPBS. They were then suspended in a solution of 1 mg/ml Collagenase Type 4 (Worthington, LS0004186) and incubated at 37° C. for 30 min, being shaken every 3 min. The reaction was stopped using DMEM media with 5% fetal bovine serum. Cell suspensions were then filtered through a 40 μm mesh filter and resuspended in 100 μl of DPBS. Cell viability was stained with GHOST DYE™ Violet 450 (Tonbo Biosciences, 13-0863-T100). Human BD Fc BLOCK™ (BD PHARMINGEN™, 564220) was used at a concentration of 5 μl/106 cells for 10 min at room temperature. Fluorescent antibody staining was done at the recommended concentrations of each antibody for 30 min at room temperature in the dark. Cell suspensions were then washed using DPBS and resuspended in 1×RBC lyse/fix solution (EBIOSCIENCE™ 00-5333-57) for 40 min. Samples were then washed in DPBS and analyzed using the BD FACSCANTO™

### Results

### Identification and Quantification of Tumor Cells Presenting Both Tumor and Lymphocyte Markers in Human RCC Samples

Macrophages have been recently implicated in abating the effects of immune checkpoint inhibitors (ICI) in treating RCC through the process of trogocytosis with cancer cells (Park, H. R., et al., Sci Rep, 2022. 12(1): p. 12546). Given that RCC is an immunogenic cancer with high levels of immune infiltration, experiments were designed to investigate if other types of immune cells may play an important role in shaping the tumor microenvironment through trogocytosis. Paraffin imbedded slides from 21 human RCC tumors of varying stages were obtained to determine whether markers of trogocytosis could be detected. Positive staining for carbonic anhydrase 9 (CAIX) was used to define tumor cells as it is a well-characterized marker of hypoxia and is commonly upregulated in RCC cells (Courcier, J., et al., Int J Mol Sci, 2020. 21(19)).

To identify trogocytic tumor cells, slides were stained with CAIX and various immune markers of interest. Fluorescent imaging revealed significant populations of cells displaying both CAIX and CD14, CD16, CD56, or CD45RA (data not shown). These results revealed that macrophages, monocytes, NK cells, and CD45RA⁺ T cells are likely the most active participants of trogocytosis with RCC cells. Interestingly, CD45RO⁺ T cells were also stained and showed little evidence of trogocytic activity when imaged. The RA isoform of CD45 is typically expressed on naïve CD4⁺ and CD8⁺ T cells, until it is converted into the RO form upon the formation of memory cells (Prince, H. E., Cellular Immunology, 1992. 145(2): p. 254-262). This implies that naïve T cells are far more trogocytic than mature T cells in RCC.

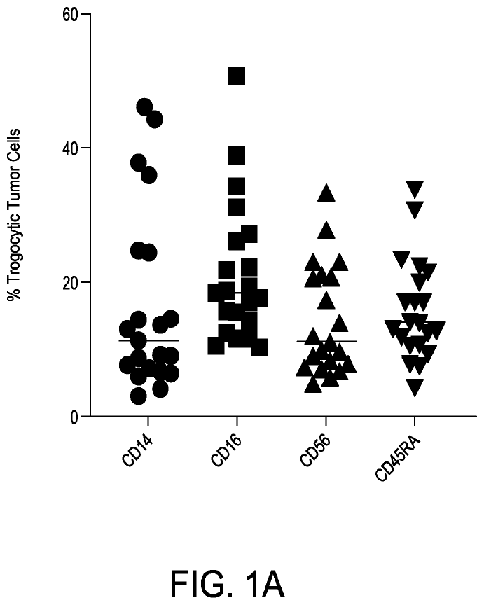

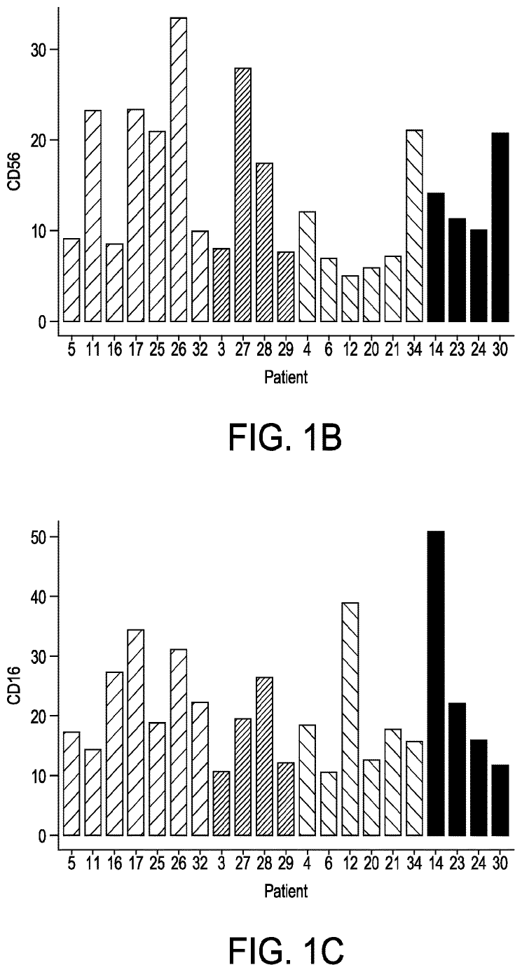

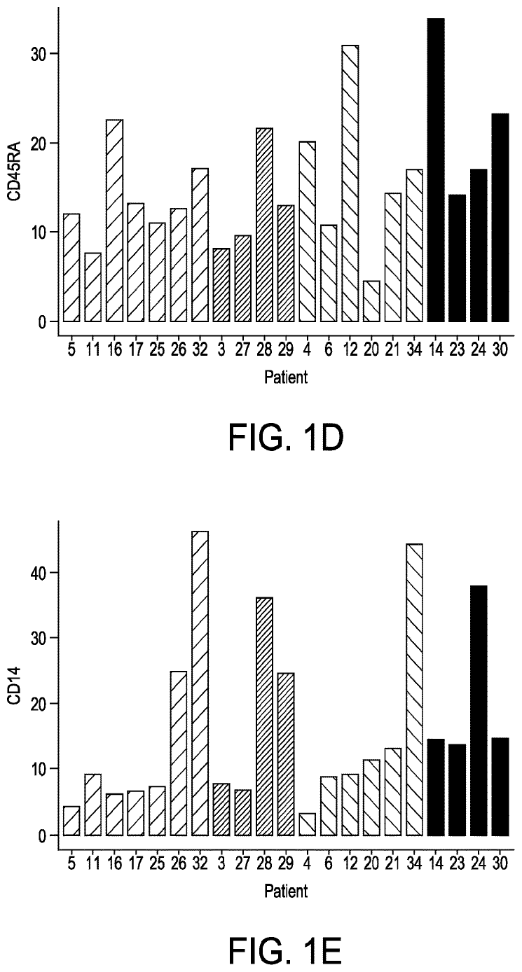

To compare the percentage of tumor cells displaying signs of trogocytosis, an image quantification program was developed that would allow the recordation of the percentage of individual cells' surface area occupied by a trogocytic marker based on IF images. Cutoffs were then established to define a tumor cell as trogo⁺ or trogo⁻ based on what percentage of their surface area was positive for a given immune marker. Through this analysis, it was found that of the 21 patient tumors images, all displayed significant signs of trogocytosis between immune cells and tumor cells (FIG. 1A). The average percentage of trogo⁺ RCC cells per tumor was between 15-20%, with some patients having tumor as high as 50% trogo⁺ for certain markers. Additionally, when tumors from different stages of disease were compared, no significant difference in the percentage of trogocytic tumor cells were found (FIGS. 1B-1E).

### Characterization of Trogocytic RCC Tumors by Flow Cytometry

Next, fresh tumors were characterized for markers of trogocytosis via flow cytometry. The IF analysis previously described has certain limitations that make distinguishing trogocytic staining from background signal difficult. Flow cytometry was chosen to provide a more accurate depiction of the trogocytic properties of RCC tumor cells. Fresh tumor samples were acquired from the University of Arizona biobanking facility and immediately dissociated and stained for markers of interest, namely CAIX, CD45, CD56, CD16, CD14, and CD68. Although CD68 was not initially included in the IF analysis due to low expression, it was concluded that difficulties detecting this antigen via fixed slide IF did not apply to fresh tissue staining. Samples were evaluated within 24 hours of receiving them to ensure maximum viability of the cells.

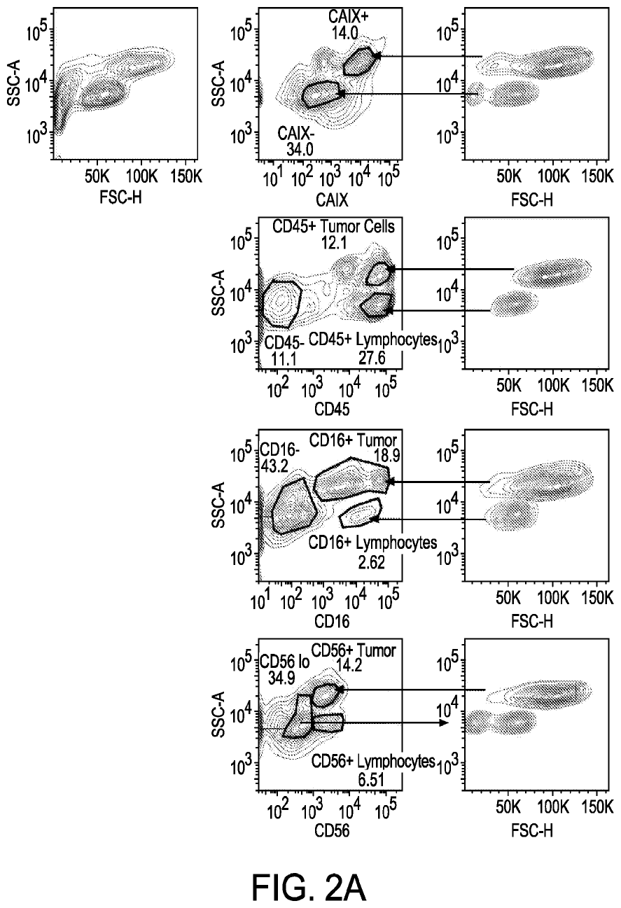

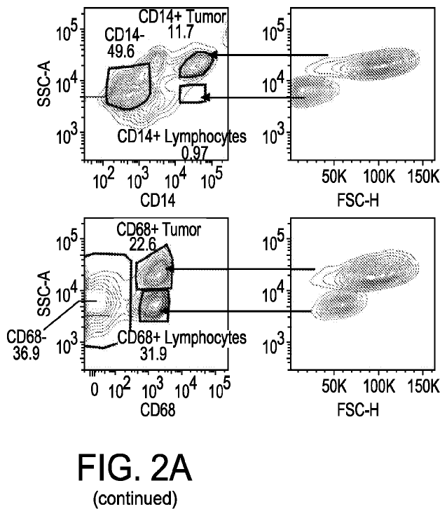

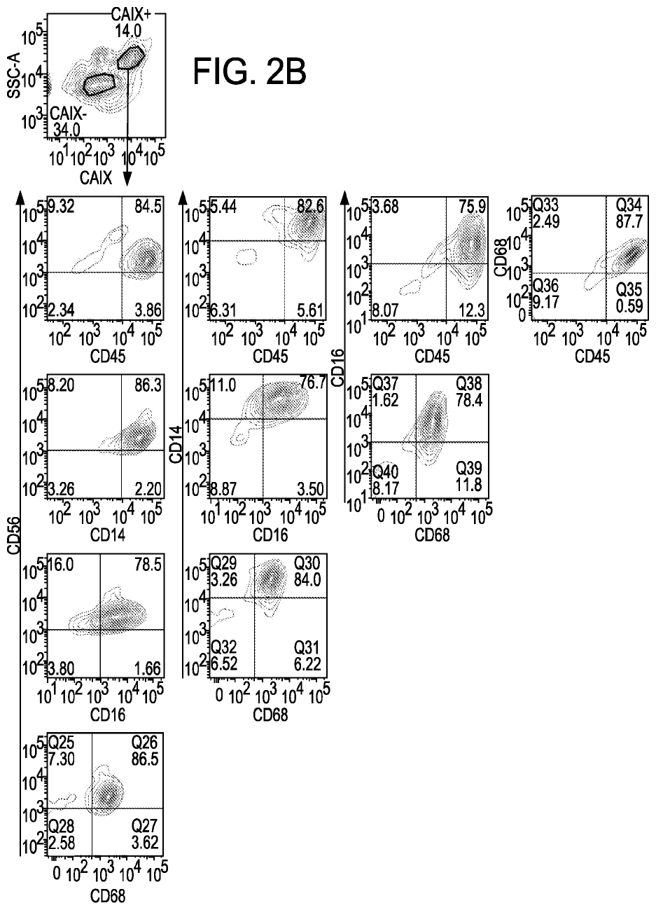

Two populations of cells were immediately apparent based on size discrimination, one presumably being tumor cells and the other being infiltrating lymphocytes (FIG. 2A). Tumor cells were identified via CAIX high staining and were confirmed to be the upper population of cells on a size discrimination plot. Both the tumor and lymphocyte populations were further characterized using a panel of trogocytic markers (CD14, CD16, CD56, CD68, and CD45).

CD45 staining revealed two populations of cells, one confirmed to be tumor cells based on CAIX high staining and size discrimination, and the other confirmed to be infiltrating lymphocytes. CD45-high cells were selected and back gated onto a size discrimination plot, which revealed significant quantities of CD45-high cells in both populations on this plot. This strategy was repeated for all immune markers of interest. CAIX-high cells were then specifically examined for the presence of these markers. Analysis revealed that 86.9% of tumor cells were also CD45 high, with an MFI comparable to that of CD45 high lymphocytes. CD16, CD56, and CD14 were also evaluated for evidence of trogocytosis and were confirmed to be present on RCC tumor cells. Of the CAIX high tumor cells, 89.3% were CD16 high, 90.2% were CD14 high, and 94.3% were CD56 high (Table 3). The data indicates that the majority of tumor cells in RCC tumors express high amounts of immune markers, and that this property is detectable by flow cytometry.

Given the high percentage of tumor cells exhibiting trogocytic markers, focus was given to characterizing cells that express multiple immune proteins. The data revealed that the vast majority of CAIX-high cells were positive for multiple immune cell markers (FIG. 2B). Therefore, it is possible that RCC tumor cells undergo multiple trogocytic events that allow them to “steal” the ability to express these markers from multiple types of immune cells.

TABLE 3
Average Percentage of Tumor Cells
Expressing Trogocytic Markers

| Patient | CD14 | CD16 | CD56 | CD45RA |
|---|---|---|---|---|
| 3 | 7.7 | 10.56 | 7.82 | 7.95 |
| 4 | 3.14 | 18.52 | 11.92 | 20.04 |
| 5 | 4.2 | 17.03 | 9.01 | 11.94 |
| 20 | 11.43 | 12.46 | 5.89 | 4.37 |
| 21 | 13.06 | 17.78 | 7.08 | 14.27 |
| 23 | 13.73 | 21.84 | 11.18 | 14.05 |
| 24 | 37.83 | 15.83 | 9.93 | 17.01 |
| 6 | 8.8 | 10.37 | 6.8 | 10.72 |
| 11 | 9.06 | 14.21 | 23.01 | 7.64 |
| 12 | 9.25 | 39.12 | 5.00 | 30.82 |
| 25 | 7.28 | 18.88 | 20.80 | 10.93 |
| 26 | 24.89 | 31.16 | 33.41 | 12.54 |
| 27 | 6.83 | 19.49 | 27.89 | 9.53 |
| 28 | 36.08 | 26.29 | 17.40 | 21.58 |
| 29 | 24.54 | 11.81 | 7.4 | 12.84 |
| 30 | 14.67 | 11.68 | 20.6 | 23.36 |
| 14 | 14.45 | 50.84 | 14.03 | 33.89 |
| 16 | 6.01 | 27.23 | 8.42 | 22.53 |
| 17 | 6.49 | 34.55 | 23.18 | 13.2 |
| 32 | 46.12 | 22.34 | 9.70 | 17.15 |
| 34 | 44.42 | 15.66 | 21.09 | 17.01 |

In this study, results showed that RCC tumor cells express significant levels of lymphoid proteins, such as CD45RA, CD14, CD16, CD56, and CD68. Importantly, this phenomenon is widespread, with over 90% of fresh RCC tumor cells showing high levels of trogocytic protein expression when analyzed via flow cytometry. This data demonstrates that RCC tumor cells acquire the ability to express large quantities of hematopoietic surface antigens following a trogocytic event with infiltrating lymphocytes.

Using immunofluorescent analysis of RCC slides, a computer algorithm was developed capable of quantifying the percentage of tumor cells in an image that express proteins found on lymphocytes. This study revealed consistent expression of the markers CD14, CD16, CD56, and CD45RA in a cohort of 21 human patient tumors when analyzed via IF. Despite limitations in the sensitivity of this method, analyzing 21 tumors originating from different stages of disease revealed no significant difference in trogocytic antigen expression at any stage of tumor development. Additionally, of the four trogocytic antigens analyzed, there was no significant changes in the specific type of antigen being expressed at any stage. These data imply that trogocytosis occurs at an early stage during tumor development, and that the trogocytic phenotype of RCC tumor cells are maintained as disease progresses. Currently, it is unknown whether this is due to repeated trogocytic interactions with infiltrating lymphocytes, acquisition of lymphocytic DNA that promotes expression of these antigens, or some combination of the two.

Analysis of fresh tumors by flow cytometry corroborated the results obtained via IF, with the addition of CD68 being frequently detected as a marker of trogocytosis. Flow cytometry revealed much higher levels of trogocytic marker expression than previously anticipated, with over 90% of tumor cells in all RCC tumor tested being positive for at least one lymphocytic marker. This is believed to be the first time these lymphocytic proteins have been identified on any solid tumor and could shift the paradigm of how a tumor's interactions with its microenvironment are understood.

Trogocytosis is an underappreciated phenomenon that shapes the immune microenvironment surrounding many types of solid tumors. The consequences of membrane-bound proteins being deposited from a donor immune cell to a recipient cancer cell via trogocytosis are still unclear. Here, results show that human kidney tumors stably express the lymphoid markers CD45, CD56, CD14, and CD16. Flow cytometry performed on fresh kidney tumors revealed consistent CD45 expression on tumor cells, as well as varying levels of the other markers mentioned previously. These results were consistent with the immunofluorescent analysis, which also revealed colocalization of lymphoid markers with carbonic anhydrase 9 (CAIX), a commonly used kidney tumor marker. Additionally, expression of these proteins is also consistent with qRT-PCR analysis. These results demonstrate that the process of trogocytosis between kidney cancer cells and infiltrating immune cells results in “hybrid cancer cells” that stably express both well characterized tumor and immune markers throughout all stages of disease. It is believed this may be true in many types of solid tumors.

Trogocytosis has been shown to promote acquisition of the immune check point receptor PD-1 by natural killer cells in mouse models of leukemia, resulting in suppressed NK cells antitumor immunity (Hasim, M. S., Science Advances, 2022. 8(15)). Additionally, colorectal cancer cells have been shown to obtain other immune regulatory molecules such as CTLA4 through trogocytosis with infiltrating lymphocytes (Shin, J. H., et al., Proc Natl Acad Sci USA, 2021. 118(48)). These results indicate that trogocytosis is a mechanism by which tumors evade elimination by the immune system, either by acquiring regulatory molecules themselves, or by indirectly benefiting from certain immune cells negatively regulating themselves. In the context of RCC, it is unknown how tumor cells use the acquisition of immune cell surface protein to promote their survival. However, given the frequent expression of these markers on RCC tumors from all stages of disease, it is believed that trogocytosis confers a survival advantage to tumor cells.

In addition to RCC tumors, several types of other solid tumors were analyzed for evidence of trogocytosis via flow cytometry. Colorectal adenocarcinomas showed expression of several trogocytic antigens including CD45, CD56, and CD16 when analyzed by IF. Widespread expression of CD45 and CD68 was identified on both colorectal and pancreatic neuroendocrine tumors via flow cytometry. These findings indicate that expression of lymphocytic antigens is a frequent occurrence on many types of solid tumors.

Given the widespread expression of trogocytic markers on RCC tumors, these antigens have significant potential as immune oncology therapy targets. Antibody-drug conjugates (ADCs) carrying a cytotoxic payload can be designed to specifically target common trogocytic motifs. This strategy can permit highly specified treatment of RCC tumor cells. Additionally, if trogocytosis is a common phenomenon in many kinds of solid tumors, these therapeutic strategies can have benefits outside of treating RCC. Trogocytosis can also be applicable to the development of improved biomarkers in solid tumors.

### Example: 2 Additional Evidence of Trogocytosis in RCC

FIG. 3A shows FACS gating strategy used to isolate lymphocyte (L), trogocytic tumor (T), and non-trogocytic tumor (NT) populations based on tumor marker (CAIX) and lymphocyte marker (CD45) discrimination. FIG. 3B shows representative data of NanoString gene expression analysis performed on human clear cell RCC tumor dissociations. Upper box indicates gene expression characteristic of lymphocyte populations, including new markers of trogocytosis such as CD4 and CD53. The lower box indicates gene expression consistent with RCC tumor cells. FIG. 3C shows CD45 qRT-PCR gene expression data of various RCC cell lines post coculture with Jurkat T cells relative to un-cocultured RCC controls.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. An antibody or antigen binding fragment comprising
i) a first antigen-binding site, and
ii) a second antigen-binding site,
wherein the first antigen-binding site specifically binds to an immune cell antigen present on a cancer cell, and the second antigen-binding site specifically binds to a non-immune cell cancer antigen, optionally wherein presence or expression of the immune cell antigen on the cancer cell is due to trogocytosis.

2. The antibody or antigen binding fragment of claim 1, wherein the cancer antigen is a cell surface molecule selected from the group consisting of 4-1BB, 5T4, adenocarcinoma antigen, alpha-fetoprotein, BAFF, BCMA, B-lymphoma cell, C242 antigen, Cancer Antigen 125 (CA-125), carbonic anhydrase 9 (CA-IX), C-MET, CCR4, CD152, CD19, CD20, CD200, CD22, CD221, CD23 (IgE receptor), CD28, CD30 (TNFRSF8), CD33, CD4, CD40, CD44 v6, CD51, CD52, CD56, CD74, CD79b, CD80, CEA, CNT0888, CTLA-4, DR5, EGFR, EpCAM, CD3, FAP, fibronectin extra domain-B, folate receptor 1, GD2, GD3 ganglioside, glycoprotein 75, glycoprotein non-metastatic B (GPNMB), HER2/neu, HGF, human scatter factor receptor kinase, IGF-1 receptor, IGF-I, IgGl, Ll-CAM, IL-13, IL-6, insulin-like growth factor I receptor, integrin α5β1, integrin ανβ3, MORAb-009, MS4A1, MUC1, mucin CanAg, N-glycolylneuraminic acid, Nectin4, NPC-1C, PDGF-R a, PDL192, phosphatidylserine, prostatic carcinoma cells, RANKL, RON, ROR1, SCH 900105, SDC1, SLAMF7, TAG-72, tenascin C, TGF beta 2, TGF-β, TRAIL-R1, TRAIL-R2, trophoblast cell surface antigen (TROP-2), tumor antigen CTAA16.88, VEGF-A, VEGFR-1, VEGFR2, and vimentin.

3. An antibody or antigen binding fragment comprising
i) a first antigen-binding site, and
ii) a second antigen-binding site,
wherein the first antigen-binding site specifically binds to an immune cell antigen present on a cancer cell, and the second antigen-binding site specifically binds to an antigen on an immune cell, optionally wherein presence or expression of the immune cell antigen on the cancer cell is due to trogocytosis, optionally wherein the cancer cell does not have the antigen on the immune cell.

4. The antibody or antigen binding fragment of claim 3, wherein the antigen on the immune cell is selected from B220/CD45R, CCR10, CCR127, CD2, CD3, CD4, CD5, CD8, CD11a, CD19, CD20, CD25, CD27, CD28, CD31, CD34, CD38, CD45RA, CD45RO, CD56, CD58, CD62L, CD103, CD122, CD161, CD194, CD195, CD196, CD197, CD294, Fc gamma RIII (CD16), Fc gamma RIIIA/CD16a, Fc gamma RIIIB/CD16b, Flt-3/Flk-2, IL-7R alpha/CD127, NCAM-1/CD56, Neprilysin/CD10, NKp46/NCR1, Siglec-2/CD22, and those listed in Table 1.

5. The antibody or antigen binding fragment of claim 3, wherein the antibody is a Bispecific T cell engager (BiTE).

6. The antibody or antigen binding fragment of claim 3, wherein immune cell antigen present on the cancer cell is a cell surface molecule selected from the group consisting of CD45, CD56, CD16, CD14, CD68, CD4, CD53, PTPRC, FCGR2A, FCGR3A, and CTSS.

7. The antibody or antigen binding fragment of claim 6, wherein the antibody or antigen binding fragment is a bispecific, trispecific, or multispecific antibody.

8. The antibody or antigen binding fragment of claim 6, wherein the antibody or antigen binding fragment is a bispecific antibody.

9. The antibody or antigen binding fragment of claim 8, wherein the antibody or antigen binding fragment is conjugated to one of more of cytotoxic agents, drugs, receptors, enzymes, receptor ligands.

10. The antibody or antigen binding fragment of claim 6, wherein the antibody or antigen binding fragment is conjugated to a cytotoxic agent.

11. The antibody or antigen binding fragment of claim 10, wherein the cytotoxic agent is selected from the group consisting of anti-microtubules agents and DNA-damaging agents.

12. The antibody or antigen binding fragment of claim 10, wherein the cytotoxic agent is selected from the from the group consisting of maytansinoids, auristatins, and calicheamicins.

13. The antibody or antigen binding fragment of claim 6, wherein the antibody or antigen binding fragment comprises an Fc fragment that facilitates binding to a Fc receptor on an immune effector cell.

14. The antibody or antigen binding fragment of claim 13, wherein the Fc receptor is FcγR.

15. The antibody or antigen binding fragment of claim 13, wherein the immune effector cell is selected from NK cells, monocytes, macrophages, neutrophils, eosinophils, and dendritic cells.

16. The antibody or antigen binding fragment of claim 13, wherein the binding to Fc receptor facilitates cytotoxic granule release, Fas signaling, elaboration of reactive oxygen species, or combinations thereof.

17. A pharmaceutical composition comprising a therapeutically effective amount of the antibody or antigen binding fragment of claim 1.

18.-31. (canceled)

32. A method of treating cancer in a subject in need thereof comprising administering to the subject an effective amount of the pharmaceutical composition of claim 17 to treat or alleviate one or more symptoms of the cancer in the subject.

33.-54. (canceled)

55. A method of identifying a compound that reduces trogocytosis and/or treats cancer comprising detecting one or more immune cell markers on cancer cells prior and after treatment with a test compound, and selecting the compound as effective for reducing trogocytosis and/or treating cancer if the immune cell markers is reduced after treatment relative to before treatment.

56. The method of claim 55 wherein the treatment is in vivo, in vitro, or ex vivo.
